(19) United States
(12) Patent Application Publication
(10) Pub. No.: US 2026/0254415 A1
(43) Pub. Date: Aug. 27, 2026

(54) GROUNDED POSITIVE AND NEGATIVE CAPACITANCE MULTIPLIER

(71) Applicant: KING FAHD UNIVERSITY OF PETROLEUM AND MINERALS, Dhahran (SA)

(72) Inventors: Ahmed Reda MOHAMED, Dhahran (SA); Muneer Ahmed AL ABSI, Dhahran (SA)

(73) Assignee: KING FAHD UNIVERSITY OF PETROLEUM AND MINERALS, Dhahran (SA)

(21) Appl. No.: 19/064,315

(22) Filed: Feb. 26, 2025

(51) Int. Cl. H03F 1/26; A61B 5/00; H03F 3/45

(52) U.S. Cl. CPC H03F 1/26; A61B 5/721; H03F 3/45183; H03F 2200/294; H03F 2203/45116

(57) ABSTRACT

A grounded positive and negative capacitance multiplier including a first number (i) of current conveyors connected in a cascaded manner. Each current conveyor includes a transconductance amplifier (OTA), a feedback network (FBN) including a second number (N) of CMOS inverters, and a feedforward network (FFN) including a third number (M) of CMOS inverters. A base capacitor is connected between a first input terminal of a first current conveyor of the cascaded current conveyors and a ground node. A multiplied capacitance is obtained between a second input terminal of the first current conveyor and the ground node, with a capacitance value given by

$${( - \frac{M}{N} )}^{i} * {C}_{B} .$$

C_{B} represents a capacitance of the base capacitor. When i is an odd number, the multiplied capacitance has a negative value, and when i is an even number, the multiplied capacitance has a positive value.

### STATEMENT REGARDING PRIOR DISCLOSURE BY THE INVENTORS

Aspects of this technology are described in an article by Muneer A. Al Absi1 and Ahmed Reda Mohamed, “Significantly Huge Positive and Negative Capacitor Multiplier” submitted to AUE Journal on Oct. 3, 2024, the content of which is herein incorporated by reference in its entirety.

### STATEMENT OF ACKNOWLEDGEMENT

Support provided by King Fahd University of Petroleum and Minerals (KFUPM) and Zagazig University (ZU) is gratefully acknowledged.

### BACKGROUND

### Technical Field

The present disclosure is directed to signal processing and, more particularly, to a grounded positive and negative capacitance multiplier and a low-pass filter for processing human biosignals.

### Description of Related Art

The “background” description provided herein is for the purpose of generally presenting the context of the disclosure. Work of the presently named inventors, to the extent it is described in this background section, as well as aspects of the description which may not otherwise qualify as prior art at the time of filing, are neither expressly or impliedly admitted as prior art against the present invention.

Biosignals have amplitudes ranging from a few microvolts to tens of millivolts. Various disturbances can significantly impact the quality of these signals. For instance, one of the most promising biosignals for early hypertension detection is the photoplethysmography (PPG) signal, which generally spans from sub-hertz to a few hertz. These biosignals can be corrupted by capacitive or inductively coupled electrical noise, such as power line interference at 50-60 Hz, or internal interference from other biosignals like electrocardiographic (ECG), electroencephalographic (EEG), electromyographic (EMG), phonocardiographic (PCG), electrogastrographic (EGG), electroretinographic (ERG), and electrooculographic (EOG) signals. As a result, a low pass filter (LPF) circuit is necessary to extract the signal while filtering out-of-band noise from external AC power interference and internal interferences. The LPF is a crucial component of a PPG sensor read-out front-end integrated circuit (IC), following the low-noise amplifier (LNA).

Since the cut-off frequency (fc) of the LPF is typically relatively small (5-10 Hz), it requires a very large resistor or capacitor, resulting in significant silicon area usage. For instance, an integrated capacitor of 100 pF occupies approximately 0.1 mm². To address this, a large capacitor that provides a long time constant while consuming minimal silicon area is often achieved using a capacitance multiplier (CM). In a CM, the basic capacitance (CB) is increased by a multiplication factor (K) through various current-mode and voltage-mode techniques.

In the voltage-mode approach, Miller's theorem can be applied to determine the capacitance multiplier, where an active amplifier with a high gain results in a large scaling factor and an increased capacitance equivalent (Ceq). However, the high gain may cause saturation in the amplifier's output, introducing undesirable distortion that limits the dynamic range. Additionally, the value of K is difficult to control precisely because the amplifier's gain depends on various factors, including process variations, supply voltage, and temperature.

In contrast, current-mode circuits can operate at lower supply voltages and exhibit greater robustness to process, voltage, and temperature (PVT) variations. The scaling factor can be precisely controlled using the transistor aspect ratio or current ratio. Various active building blocks (ABBs) can be used in current-mode capacitance multipliers to achieve a large multiplication factor (K), including, but not limited to, operational transconductance amplifiers (OTA), differential-output OTAs (DO-OTA), second-generation current conveyors (CCII), super flipped current followers (SFCF), current-feedback operational amplifiers (CFOA), current-controlled current differencing transconductance amplifiers (CCCDTA), inverting second-generation current conveyors (ICCII), current follower transconductance amplifiers (CFTA), current difference transconductance amplifiers (CDTA), and current-feedback operational amplifiers (CFOA).

For instance, a capacitor multiplier-based current-mode circuit with a positive floating and tunable CM configuration utilizing four active devices (a single CCII, two OTAs, and a single DO-OTA) consumes only 2.3 μW over a limited frequency range of 5 kHz. However, the maximum value of K is 3600, with a maximum error of 8.6%, attributed to the use of a single-stage OTA structure, which limits the open-loop gain. A grounded capacitor multiplier based on CFOAs achieves a K of 1000 while consuming 63 μW. However, the circuit's use of floating and grounded passive resistors increases silicon area and power consumption.

An adjustable grounded CM based on CCCDTA consumes approximately 600 μW with a limiting K of 150 and operates in a frequency range of 10 kHz to 10 MHz, making it suitable for speech processing. A negative CM circuit based on a single CFOA achieves a K of 500 but it requires two passive resistors, resulting in a larger silicon area and significant power consumption of 1.6 mW within an operating span of 80 MHz. A CM circuit using a single CDTA implements a K of 300 and consumes 720 μW. However, low-power CM circuits with K values of 318 use a floating capacitor as the base capacitor. Low-power CMs typically require double-poly processes in integrated circuits (ICs) and consume power equal to or greater than 500 μW, with a limited multiplication factor of less than 1000.

Thus, figures of merit (FOMs) are essential for evaluating the functional performance and integration complexity of these circuits. FOMs may include parameters such as multiplication factor (K), supply voltage (SV), power dissipation (PD), bandwidth (BW), total number of active devices (AD), total number of passive resistors (PR), total number of transistors (NT), and base capacitance (CB). To address these challenges, there is a need to develop current multipliers that close the design gaps and provide clear pathways for improvement.

Conventional capacitor multiplier implementations suffer from limitations such as a multiplication factor of less than 1000, reliance on floating capacitors, use of multiple active devices with passive resistors, limited accuracy, and high power consumption (greater than 500 μW).

Accordingly, it is one object of the present disclosure to provide the capacitor multiplier for overcoming the limitations of conventional approaches by offering a grounded positive and negative capacitance multiplier used for processing human biosignals.

### SUMMARY

In an exemplary embodiment, a grounded positive and negative capacitance multiplier is disclosed. The grounded positive and negative capacitance multiplier comprises a first number (i) of current conveyors connected in a cascaded manner. Each current conveyor includes a transconductance amplifier (OTA), a feedback network (FBN) including a second number (N) of CMOS inverters, and a feedforward network (FFN) including a third number (M) of the CMOS inverters. A base capacitor is connected between a first input terminal of a first current conveyor of the cascaded current conveyors and a ground node. A multiplied capacitance is obtained between a second input terminal of the first current conveyor and the ground node with a capacitance value given by

$${( - \frac{M}{N} )}^{i} * {C}_{B} ,$$

where C_{B} represents a capacitance of the base capacitor. When i is an odd number, the multiplied capacitance has a negative value, and when i is an even number, the multiplied capacitance has a positive value.

In another exemplary embodiment, a low-pass filter used for processing human biosignals is disclosed. The low-pass filter comprises a multiplied capacitance obtained from a base capacitor and a grounded positive and negative capacitance multiplier. The capacitance multiplier includes a first number (i) of current conveyors connected in a cascaded manner. Each current conveyor includes a transconductance amplifier (OTA), a feedback network (FBN) including a second number (N) of CMOS inverters, and a feedforward network (FFN) including a third number (M) of CMOS inverters. The base capacitor is connected between a first input terminal of a first current conveyor of the cascaded current conveyors and a ground node. The multiplied capacitance is obtained between a second input terminal of the first current conveyor and the ground node, with a capacitance value given by

$${( - \frac{M}{N} )}^{i} * {C}_{B} ,$$

where C_{B} represents a capacitance of the base capacitor. When i is an odd number, the multiplied capacitance has a negative value, and when i is an even number, the multiplied capacitance has a positive value.

The foregoing general description of the illustrative embodiments and the following detailed description thereof are merely exemplary aspects of the teachings of this disclosure, and are not restrictive.

### BRIEF DESCRIPTION OF THE DRAWINGS

A more complete appreciation of this disclosure and many of the attendant advantages thereof will be readily obtained as the same becomes better understood by reference to the following detailed description when considered in connection with the accompanying drawings, wherein:

FIG. 1A illustrates a graph diagram representing frequency spectrum of a plurality of biosignals, according to certain embodiments.

FIG. 1B illustrates a block diagram of a photoplethysmograhic (PPG) sensor front-end circuit, according to certain embodiments.

FIG. 2A illustrates a graph diagram representing a comparison of a figure of merits (FOM₁) vs. FOM₂.

FIG. 2B illustrates a graph diagram representing a comparison of FOM₃ VS. FOM₄.

FIG. 3A illustrates an exemplary schematic diagram of a modified 2nd generation current conveyor (M-CCII), according to certain embodiments.

FIG. 3B illustrates an exemplary schematic diagram of the M-CCII, according to certain embodiments.

FIG. 4 illustrates an exemplary circuit diagram of the M-CCII, according to certain embodiments.

FIG. 5 illustrates an exemplary block diagram representation of a capacitance multiplier including a cascaded connection of a plurality of M-CCII blocks, according to certain embodiments.

FIG. 6A illustrates a graph diagram representing the multiplication factor with respect to the number of M/N and the number of i, according to certain embodiments.

FIG. 6B illustrates a graph diagram representing an estimated power consumption with respect to the number of M/N and the number of i, according to certain embodiments.

FIG. 7 illustrates an exemplary flow diagram of an architecture-based design methodology, according to certain embodiments.

FIG. 8A illustrates an exemplary schematic diagram of M-CCII (with M=15, N=1), according to certain embodiments.

FIG. 8B illustrates an exemplary circuit diagram of M-CCII (with M=15, N=1), according to certain embodiments.

FIG. 8C illustrates an exemplary stable current reference circuit according to certain embodiments.

FIG. 9 illustrates an exemplary flow diagram of a circuit-based design methodology, according to certain embodiments.

FIG. 10 illustrates a graph diagram representing the input-output transfer characteristics of the conventional and the modified CMOS inverters, according to certain embodiments.

FIG. 11 illustrates an exemplary schematic diagram of the capacitance multiplier (CM)-based M-CCII, according to certain embodiments.

FIG. 12 illustrates a graph diagram representing a frequency response to a voltage gain, according to certain embodiments.

FIG. 13 illustrates a graph diagram representing a frequency response to a current gain, according to certain embodiments.

FIG. 14 illustrates a graph diagram representing impedances at X, Y, and Z terminals, according to certain embodiments.

FIG. 15 illustrates a graph diagram representing impedance of the M-CCII-based CM, according to certain embodiments.

FIG. 16 illustrates a graph diagram representing frequency resonance of the low-pass-filter (LPF), according to certain embodiments.

FIG. 17A illustrates a graph diagram representing frequency response of the LPF based on the M-CCII-based capacitor multiplier, according to certain embodiments.

FIG. 17B illustrates a graph diagram representing bandwidth of the LPF within PVT variations, according to certain embodiments.

FIG. 18 illustrates a graph diagram representing Monte Carlo results of the LPF's bandwidth, according to certain embodiments.

FIG. 19 illustrates a graph diagram representing a noisy PPG signal and the LPF's output, according to certain embodiments.

FIG. 20A and FIG. 20B illustrate graph diagrams representing a comparison of the CCII-based CM and multiple prior arts in terms of FOSM₁ vs. FOM₂ and FOM₃ VS. FOM₄, respectively.

FIG. 21 shows an illustration of a non-limiting example of details of computing hardware used in the computing system, according to certain embodiments.

FIG. 22 shows an exemplary schematic diagram of a data processing system used within the computing system, according to certain embodiments.

FIG. 23 shows an exemplary schematic diagram of a processor used with the computing system, according to certain embodiments.

FIG. 24 shows an illustration of a non-limiting example of distributed components which may share processing with the controller, according to certain embodiments.

### DETAILED DESCRIPTION

In the drawings, like reference numerals designate identical or corresponding parts throughout the several views. Further, as used herein, the words “a,” “an,” and the like generally carry a meaning of “one or more,” unless stated otherwise.

Furthermore, the terms “approximately,” “approximate,” “about,” and similar terms generally refer to ranges that include the identified value within a margin of 20%, 10%, or preferably 5%, and any values therebetween.

Aspects of this disclosure are directed to a grounded positive and negative capacitor multiplier (CM) with a very high multiplication factor. The CM can be used to implement low pass filters in biosignal interfaces, such as photoplethysmograhic (PPG) sensors. The low pass filters can effectively filter out 50-60 Hz power line frequency and internal signal interferences in the sensed biosignals.

The present disclosure relates to a grounded positive and negative capacitor multiplier comprising current conveyors connected in a cascaded manner. The grounded positive and negative CM is designed using modified 2nd-generation current conveyors (M-CCII) with a grounded capacity. Using a base capacitor of 1 pF, the equivalent capacitance reaches up to 50,625 pF with a maximum error of 2.3% while consuming a relatively low power of 250 μw.

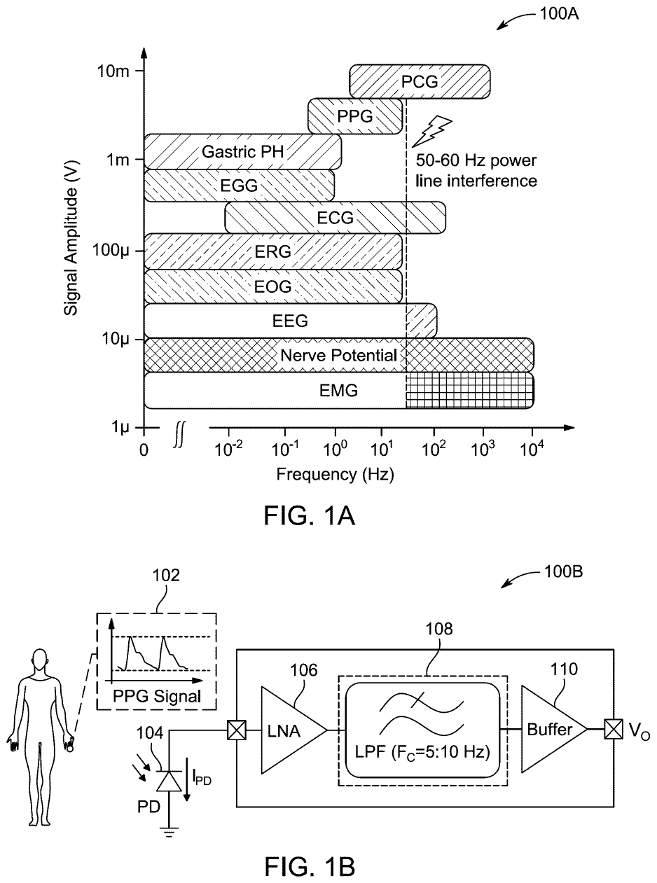

FIG. 1A illustrates a graph diagram (100A) representing frequency spectrum of a plurality of biosignals, according to certain embodiments.

The illustrated plurality of biosignals comprise a photoplethysmography (PPG) signal, electrocardiographic (ECG), electroencephalographic (EEG), electromyographic (EMG), phonocardiographic (PCG), electrogastrographic (EGG), electroretinographic (ERG), and electrooculographic (EOG).

The photoplethysmography (PPG) biosignal refers to the electrical or digital signal generated from measuring blood volume changes in a microvascular tissue, typically using light-based sensors. The PPG biosignal is captured by a photodetector, which measures the reflected or transmitted light from the skin after it is illuminated by a light source (e.g., infrared or visible light). As the heart beats, the blood volume in the vessel's changes, causing fluctuations in the amount of light reflected back to the sensor, producing a time-varying signal.

The electrocardiographic (ECG) biosignal refers to the electrical activity of the heart, recorded via electrodes placed on the skin. The ECG biosignal reflects the electrical impulses that trigger the heart's contractions, providing valuable information about the heart's rhythm, rate, and overall electrical function.

The electroencephalographic (EEG) biosignal refers to the electrical activity generated by the brain, which is recorded through electrodes placed on the scalp. These signals represent the brain's electrical impulses, primarily produced by the synchronous firing of neurons. The EEG biosignal is typically measured in the form of waveforms that vary in frequency, amplitude, and pattern, and these waveforms correspond to different mental states and activities, such as wakefulness, sleep, relaxation, or concentration.

The electromyographic (EMG) biosignal refers to the electrical activity generated by muscle fibers during contraction, which is measured using electrodes placed on the skin (surface EMG) or directly inserted into the muscle (intramuscular EMG). These electrical signals result from the depolarization of muscle fibers when they contract and are captured as a time-varying signal that reflects muscle activity.

The phonocardiographic (PCG) biosignal refers to the sound recordings generated by the mechanical activity of the heart, typically captured through a microphone or sensor placed on the chest. These sounds are produced by the heart valves closing and the blood flow through the heart and blood vessels.

The electrogastrographic (EGG) biosignal refers to the electrical activity of the stomach, typically recorded using electrodes placed on the surface of the abdomen. The EGG biosignal captures the slow, rhythmic electrical waves generated by the smooth muscle cells of the stomach during digestion, known as gastric myoelectrical activity. These electrical signals help regulate the contractions of the stomach muscles, which are essential for moving food through the digestive tract.

The electroretinographic (ERG) biosignal refers to the electrical response generated by the retina when it is stimulated by light. This signal is typically recorded by placing electrodes on the surface of the cornea or the skin around the eye. The ERG measures the retinal cells' electrical activity, including the responses of the photoreceptors (rods and cones), bipolar cells, and other retinal neurons to light stimuli.

The electrooculographic (EOG) biosignal refers to the electrical activity generated by the movement of the eyes. It is recorded using electrodes placed around the eyes, typically near the outer canthi or on the forehead, to detect the potential differences that occur when the eye moves. The eye generates a natural electrical potential due to the difference between the cornea (positive) and the retina (negative), which changes as the eye shifts its position.

As illustrated in FIG. 1A, the x-axis of the graph 100A represents frequency (Hz) and the y-axis represents signal amplitude (V) of the plurality of biosignals. The plurality of biosignals has amplitudes between a few microvolts and tens of millivolts. Various disturbances can significantly impact the signals' quality. For example, one of the most interesting biosignals for hypertension early detection, the photoplethysmography (PPG) signal, typically spans from sub-hertz to a few hertz. The bio signals can be corrupted due to capacitive or inductively linked electrical noise by a power line interference at 50-60 Hz or by the internal interferences due to other biosignals.

FIG. 1B illustrates a block diagram 100B of a photoplethysmograhic (PPG) sensor front-end circuit, according to certain embodiments.

As shown in FIG. 1B, the PPG sensor front-end circuit comprises a photodiode (PD) 104, a low-noise amplifier (LNA) 106, a low-pass filter (LPF) 108, and a buffer 110.

The photodiode 104 is a semiconductor device that converts light into an electrical current. It operates based on the photoelectric effect. In the photodiode 104, the semiconductor material absorbs photons (light particles), generating electron-hole pairs that result in a flow of current. The amount of current generated is directly proportional to the intensity of the incident light.

The LNA 106 is an electronic device designed to amplify weak signals while introducing as little additional noise as possible. The key function of the LNA 106 is to increase the strength of a signal, such as one received from a sensor, without significantly degrading the signal-to-noise ratio (SNR). The LPF 108 is an electronic circuit or a signal processing tool that allows low-frequency signals to pass through while attenuating (reducing the strength of) higher-frequency signals. The LPF 108 filters out unwanted high-frequency noise or interference.

The buffer 110 refers to an intermediary circuit or a component used to temporarily store and manage data or signals between other components of the circuit.

As shown in FIG. 1B, the LPF 108 is a building block of a PPG sensor read-out front-end integrated circuit after the LNA 106. The low pass filter (LPF) circuit reads out the signal and filters the out-of-band noise caused by external AC power noise and internal interferences.

The LPF 108 requires a very large resistor or a large capacitor, as the cut-off frequency (FC) of the LPF 108 needs to be significantly small (5-10 Hz). However, a large capacitor typically occupies a large silicon area because an integrated capacitor of 100 pF would need about 0.1 mm². In order to implement the large capacitor for providing a long-time constant while consuming a minimal silicon area, the capacitance multiplier (CM) can be used in the PPG sensor. As in the CM, the basic capacitance (CB) value is increased by a multiplication factor (K) using various methods leveraging current-mode and voltage-mode strategies.

In the voltage mode, Miller's theorem may be used to determine the CM. An active amplifier with a high gain may lead to a large scaling factor and increased capacitance equivalent (Ceq.). However, the high gain causes the amplified stage's output to saturate and produce undesirable distortion, which limits the dynamic range. Further, the value of K cannot be precisely identified because the gain value of the amplifier depends on several parameters (e.g., processes, supply voltage, and temperature).

Conversely, the current-mode circuit may function at low supply voltages and exhibit remarkable robustness to PVT variations. Furthermore, the scaling factor can be identified precisely utilizing the transistor aspect ratio or the current ratio of the current mode circuit. In the current-mode capacitance multipliers, different active building blocks (ABB) may be used to achieve the large K. The ABB includes, but is not limited to, an operational transconductance amplifier (OTA), a differential-output OTA (DO-OTA), 2nd generation current conveyor (CCII), a super flipped current follower (SFCF), a current-feedback operational amplifier (CFOA), a current controlled current differencing transconductance amplifier (CCCDTA), an inverting second-generation current conveyor (ICCII), a current follower transconductance amplifier (CFTA), a current difference transconductance amplifier (CDTA), and a current-feedback operational amplifier (CFOA).

The OTA refers to an amplifier that converts an input voltage signal into an output current. The OTA provides a current output that is proportional to the input voltage, with the proportionality factor being a transconductance denoted as ‘gm’.

The CCCDTA is an advanced analog circuit that combines functions (e.g., current differencing, transconductance amplification, and current control). The CCCDTA is a type of current-mode amplifier that operates on current signals. The CCCDTA is a versatile building block used in various analog signal processing applications (e.g., filters, oscillators, and amplifiers). The ICCII refers to a type of current conveyor that operates as a current-mode active device. The ICCII is a variation of the CCII but with an inverting configuration at the output. The ICCII is a useful building block in analog signal processing in circuits where both current and voltage signals need to be handled efficiently, such as in filters, amplifiers, and oscillators. The DO-OTA refers to a type of OTA with two output terminals, each providing a current proportional to the difference in voltage between its two input terminals. The CCII is an electronic building block used in analog signal processing. The CCII transfers voltage and current with specific relationships between inputs and outputs.

The CCII is an advanced type of current conveyor used in analog signal processing and active circuit design. The CCII is basically an extension of the original current conveyor concept, offering improved performance and flexibility, especially in current-mode applications. In an aspect, a Flipped Current Follower (FCF) is a type of current-mode analog circuit that replicates or follows an input current at its output. The FCF is a variation of the current follower circuit. The FCF produces an output current that is proportional to the input current. The FCF has a flipped configuration of the circuit, where the input current is provided at one terminal (typically at the Y input) and the output current is taken from another terminal (typically at the X output).

The Super Flipped Current Follower (SFCF) is a specialized current-mode analog circuit. The SFCF is an enhancement of the FCF. The SFCF provides accurate current-following behavior while improving various performance parameters, such as linearity, input impedance, and output impedance. The SFCF is used in applications, such as analog signal processing, filters, and current-mode circuits. The Current-Feedback Operational Amplifier (CFOA) refers to a type of operational amplifier (op-amp) that is designed to offer high-speed performance with specific benefits in certain analog circuits. The CFOAs operate based on a feedback current that controls the output current. The Current Follower Transconductance Amplifier (CFTA) is an active analog circuit that combines the properties of a current follower and a transconductance amplifier. The CFTA is a type of current-mode amplifier that processes signals in terms of currents rather than voltages. The CFTA is a versatile building block used in various analog signal processing applications (e.g., filters, amplifiers, and oscillators).

The Current Difference Transconductance Amplifier (CDTA) is an active device used in analog signal processing. The CDTA has two inputs, one for the voltage (voltage-controlled input) and one for the current (current-controlled input), and three outputs (one voltage output and two current outputs). The voltage output is related to the difference in the currents applied at the current input. Two current outputs are related to the input voltage and the current difference. The voltage input controls the current output, where the output current is proportional to the voltage input, modified by a transconductance factor. The current input produces a current difference, which helps drive the other outputs.

Conventional capacitor multiplier-based current-mode circuit may have different configurations.

For example, a positive floating and tunable CM comprises four active devices i.e., a single CCII, two OTAs, and a single DO-OTA. The positive floating and tunable CM consumes a low power of 2.3 μW over a limited frequency range of 5 kHz. However, the maximum value of K is 3600, which results in a maximum error of 8.6% due to the use of a single-stage structure OTA, which provides a limited open-loop gain.

A grounded capacitor multiplier is based on CFOAs with a K of 1000 and consumes a power of 63 μw. The grounded capacitor multiplier uses a floating and grounded passive resistor, which would waste silicon area and power.

An adjustable grounded CM based on CCCDTA has a power consumption of 600 μw with a limiting K of 150. The operating frequency of the adjustable grounded CM is noticeably from 10 kHz to 10 MHz for speech processing implementation.

A negative CM circuit based on a single CFOA has a K of 500. The negative CM circuit requires a large silicon area as it uses four passive resistors. As a result, the negative CM circuit consumes a significant power of 1.6 mW while achieving a limited value of K up to 500 within an operating span of 80 MHz

A single CDTA has a K of 300 and consumes a significant power of 720 μw. A low-power CM has a K of 318.

The voltage-mode CM and the current-mode CMs with the ABBs (e.g., OTA, DO-OTA, CCII, CCCDTA, CFTA, and CDTA) use a floating capacitor as a base capacitor. So, the voltage-mode CM and current-mode CMs with the ABBs (e.g., OTA, DO-OTA, CCII, CCCDTA, CFTA, and CDTA) require double poly in the integrated circuit (IC) process. The CCCDTA, the CFOA, and the CDTA consume power equal to and more than 500 μw, while achieving a limited multiplication factor of less than 1000.

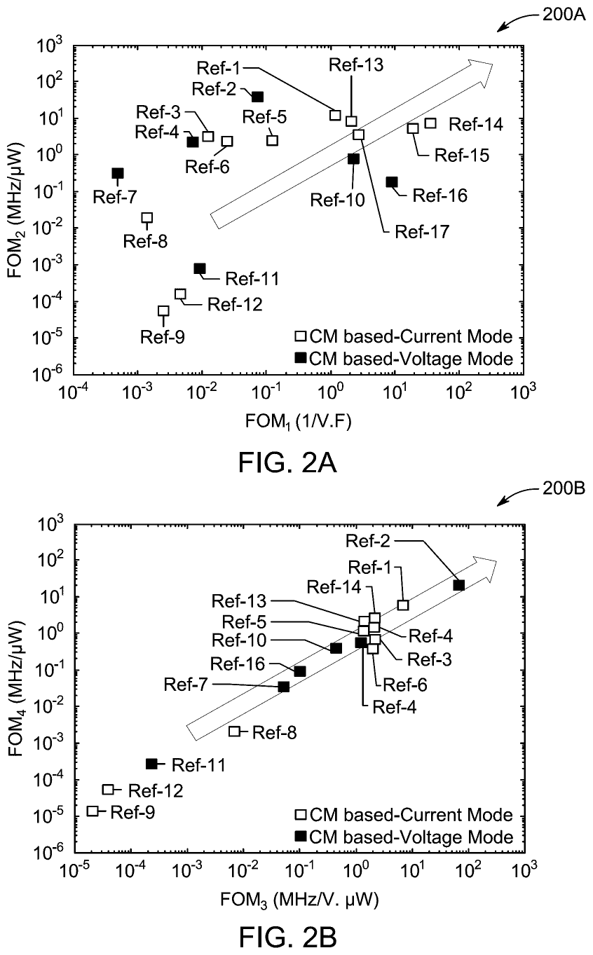

FIGS. 2A and 2B illustrate graph diagrams 200A, 200B representing a comparison of multiple prior arts in terms of a figure of merits (FOM₁) vs. FOM₂ and FOM₃ VS. FOM₄, respectively.

In an aspect, a Figure of Merit (FOM) is a quantitative measure used to assess the overall performance or efficiency of a device, system, or component in a particular application/circuit. The FOM combines multiple characteristics into a single value or a set of values that reflects the trade-offs between various performance factors.

In an operative aspect, the Figures of merit (FOMs) are used to assess the functional performance and integrations' complexity. The FOMs represent design parameters, such as multiplication factor (K), supply voltage (SV), power dissipation (PD), bandwidth (BW), the total number of active devices (AD), the total number of passive resistors (PR), and the total number of transistors (NT) and base capacitor (CB), etc.

The multiplication factor represents a factor by which one value is multiplied to yield another value. Further, the multiplication factor describes the ratio by which a certain quantity is increased or scaled. It is a numerical value that indicates how much larger or smaller the output is compared to the input.

The supply voltage refers to the electrical potential difference (voltage) provided to a circuit or electronic device by a power source (e.g., battery, power supply, or generator) to drive its operation. The supply voltage is the voltage level that powers the components of the circuit to enable them to function properly.

The bandwidth refers to the range of frequencies over which a system or a component can effectively operate, transmit, or process signals.

The active devices refer to components that can amplify a signal or control the flow of current using an external power source. The active devices are capable of injecting power into the circuit and often can change the magnitude or direction of the signal.

The power dissipation refers to electrical energy that is converted into heat and lost in an electronic component as a result of its operation. The power dissipation occurs whenever the electrical current flows through resistive or other components (e.g., transistors, resistors, diodes, etc.), causing energy to be lost as heat rather than being fully utilized for the desired function (e.g., amplification, computation, or signal processing).

The passive resistor refers to a two-terminal electronic component that resists the flow of electric current, causing a voltage drop across its terminals according to Ohm's Law. The passive resistor does not require an external power supply to function and does not amplify or control current. It dissipates energy in the form of heat as electrical energy flows through it.

The base capacitor refers to a capacitor physically connected to the capacitance multiplier (CM). The capacitance of the base capacitor is increased by the CM's multiplication factor, providing a significantly larger equivalent capacitance.

As illustrated in FIG. 2A, the x-axis of the graph 200A represents FOM₁ (1/V·F) and the y-axis represents FOM₂ (MHz/μW). The FOM₁ against the FOM₂ shows the current CMs and highlights design gaps.

As illustrated in FIG. 2B, the x-axis of the graph 200B represents FOM₃ (MHz/V·μW) and the y-axis represents FOM₄ (MHz/μW). The FOM₃ is displayed against FOM₄.

Conventional methods implementing the capacitor multiplier have many challenges, such as a limited multiplication factor of less than 1000, less accuracy, and high-power consumption of more than 500 μw.

The present disclosure discloses a grounded positive and negative capacitance multiplier for biosignal interface integrated circuit. The CM is implemented using a CCII with a grounded capacity. The CM uses a base capacitor of 1 pF; the resulting equivalent capacitance may reach up to 50,625 pF with a maximum error of 2.3% while consuming a relatively low power of 250 μw. The redesign cycle time is also reduced for the CM. Monte Carlo simulations and process-voltage-temperature (PVT) variations may be used to assess the whole performance.

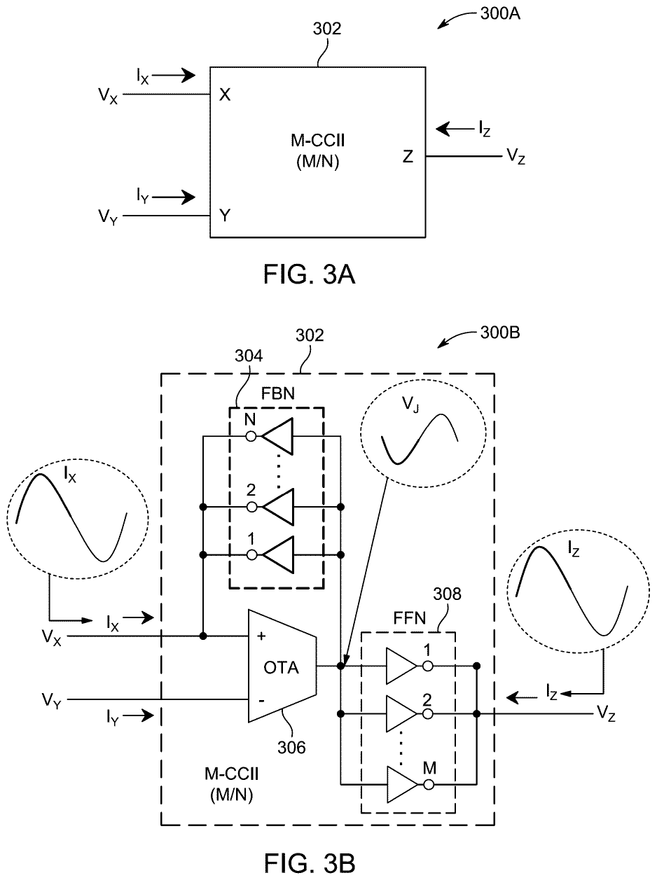

FIG. 3A illustrates an exemplary schematic diagram 300A of a modified second-generation current conveyor (M-CCII) 302, according to certain embodiments.

As shown in FIG. 3A, the M-CCII 302 is a three-terminal device. The three terminals comprise two input terminals (X, Y) and one output terminal (Z). The input terminals X and Y have voltage notation of V_{X} and V_{Y} and current notation of I_{X} and I_{Y}, respectively. An output terminal Z have voltage and current notions of V_{Z} and I_{Z}, respectively.

FIG. 3B illustrates an exemplary schematic diagram 300B of the M-CCII 302, according to certain embodiments.

As shown in FIG. 3B, an internal structure of the M-CCII 302 comprises a single transconductance amplifier (OTA) 306, a feedback network (FBN) 304, and a feedforward network (FFN) 308. An array of N CMOS and M CMOS inverters are used to build the FBN 304 and FFN 308, respectively. The FBN 304 is connected between the OTA's output and its non-inverting terminal. A feedback loop is functioned as negative feedback due to the phase shift provided by the CMOS inverter.

In an operative aspect, the injected current (Ix) is converted into a compressed voltage at a joint voltage node (V_{J}), with a phase shift of 180°, through the FBN 304. Then, the compressed voltage is transferred to a current (I_{Z}) through the FFN 308. The current I_{Z} is equal to the current Ix multiplied by a ratio of M divided by N when keeping V_{Z} equal to V_{X}. The FBN 304 contains a nonlinear element resulting in a distortion at the joint voltage node (V_{J}). This phenomenon can be reversed through the FFN 308, by guaranteeing both FBN 304 and FFN 308 are matched enough.

For example, to suppress mismatch between the inverters in the FBN 304 and FFN 308, two main strategies can be executed during the schematic design and the layout design phases. The first strategy applies to the schematic design phase, while the second applies to the layout design phase.

It has been observed that the standard deviation of the mismatch of a generic parameter, P,

$$( {σ}_{P} = \frac{{A}_{P}}{\sqrt{W * L}} )$$

is inversely proportional to the square root of the MOSFET's area, according to the Pelgrom model. The generic parameter can be a threshold voltage (Vₜₕ) or process gain factor (β). Here, A_{P} is the area proportionality constant of the parameter and is dependent on the technology used. L and W are the length and width of the transistor. Monte Carlo statistical analysis can be utilized to select the proper MOSFET's dimensions to guarantee the target operation and minimize the mismatching.

For instance, W and L can be selected as 2 μm and 5 μm, respectively. L is chosen to be large enough to reduce the consumed power, meanwhile W*L=10 μm² assists in the reduction of the standard deviation. Table 2 below provides μ, σ, and coefficient variance

$$( C V = \frac{σ}{μ} * 100 )$$

for the threshold voltage of NMOS and PMOS that can be used in the design of the inverters in the FBN and FFN.

TABLE 2
Statistical Variations Based On Monte Carlo Analysis

| NMOS's threshold | | | | PMOS' threshold | | |
|---|---|---|---|---|---|---|
| W*L | | | | \| μ \| | \| σ \| | |
| (μm²) | μ(mV) | σ(mV) | CV(%) | (mV) | (mV) | CV(%) |
| 1 | 481.3 | 10.89 | 2.26 | 448.3 | 11.45 | 2.55 |
| 5 | 458.9 | 10.24 | 2.23 | 437.6 | 10.99 | 2.51 |
| 10 | 459.9 | 8.747 | 1.90 | 445.3 | 10.08 | 2.26 |

Moreover, in analog integrated circuits, the layout significantly impacts a circuit's matching qualities; thus, some crucial factors are taken into account, as follows:
1. MOSFET's length and width are usually larger than the feature-length of the technology by at least three times. Hence, local variations of the MOSFET's dimension would be ceased.
2. To keep both the FBN and FFN matched and isolate them from the substrate noise, N+ and P+ guard rings are exploited around PMOS and NMOS transistors.
3. Cross-wiring is avoided, and keeping wires shorter between the FBN and FFN networks to get a good block-driven floor planning.

As a result, the total mismatch of the MOSFETs and the mismatch between both FBN and FNN decreased significantly during the schematic and layout design phases.

In the OTA 306 configuration with negative feedback, the V_{X} follows the V_{Y} as the open loop gain is large enough. The I_{Y} equals zero as the internal structure of the OTA 306 is CMOS-based.

Hence, the M-CCII's characteristic is given by an Equation (1) defined below:

$$[ \begin{matrix}{I}_{z} \\ {V}_{x} \\ {I}_{y}\end{matrix} ] = [ \begin{matrix}0 & β & 0 \\ & α00 & \\ & 000 & \end{matrix} ] [ \begin{matrix}{V}_{y} \\ {I}_{x} \\ {V}_{z}\end{matrix} ] \qquad ( 1 )$$

where, β and α represent the current and the voltage gain, respectively.

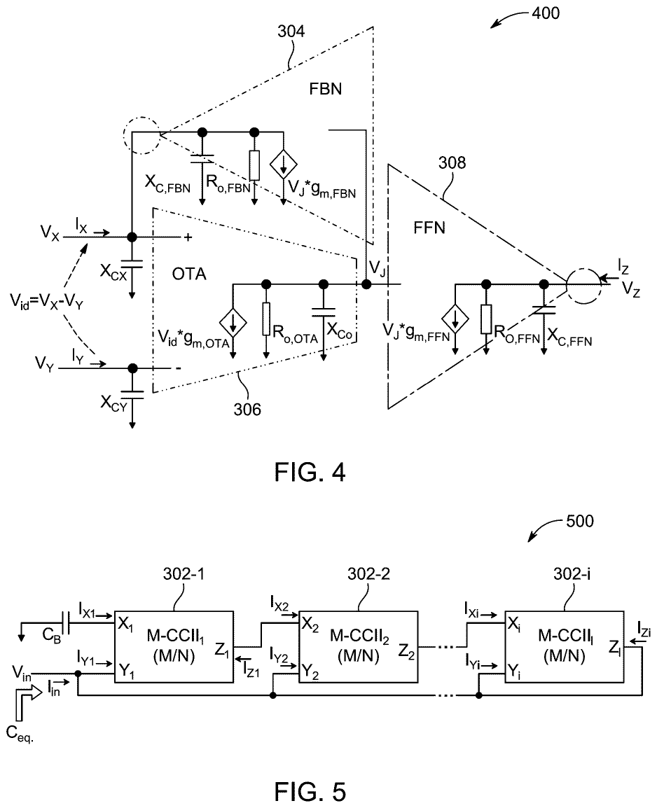

FIG. 4 illustrates an exemplary circuit diagram 400 of the M-CCII 302, according to certain embodiments.

The non-ideal behavior model of the M-CCII 302 is examined using the small signal equivalent circuit shown in FIG. 4.

The small signal equivalent circuit 400 comprises three circuits having X, Y, and Z ports. The impedance at the X, Y, and Z ports is Z_{X}, Z_{Y}, and Z_{Z}, respectively. α and β can be expressed by equations (2) and (3), respectively.

$$α = \frac{{g}_{m , FBN} * {g}_{m} * {R}_{k} \vert \vert {X}_{Co}}{\frac{1}{{X}_{Cx} ‖ {X}_{C , FBN} ‖ {R}_{o , FBN}} + {g}_{m , FBN} * {g}_{m} * {R}_{o} \vert \vert {X}_{Co}} \qquad ( 2 )$$

$$β = \frac{{I}_{Z}}{{I}_{X}} \tilde{=} \frac{{g}_{m , FFN}}{{g}_{m , F B N}} \qquad ( 3 )$$

The impedances Z_{X}, Z_{Y}, and Z_{Z} can be derived using equations (4), (5), and (6), respectively.

$${Z}_{X} = \frac{1}{\frac{1}{{X}_{C x} ‖ {X}_{C , F B N} ‖ {R}_{o , F B N}} + {g}_{m , FBN} * {g}_{m} * {R}_{o} \vert \vert {X}_{C o}} \qquad ( 4 )$$

$${Z}_{Y} = \frac{1}{2 π {C}_{Y}} \qquad ( 5 )$$

$${Z}_{Z} = {X}_{C , F F N} \vert \vert {R}_{o , F F N} \qquad ( 6 )$$

where gₘ and Rₒ are the transconductance and the output resistance of the OTA 306, respectively;
g_{m,FBN}(FFN) is the total transconductance of the feedback network (feedforward network);
R_{o, FBN}(FFN) is the total output resistor of the feedback network (feedforward network);
X_{C, FBN}(FFN) is the total output capacitive impedance of the feedback network (feedforward network);
X_{Co} is the total output capacitive impedance of the OTA 306 including the input capacitive impedance of the FBN 304 and FFN 308;
X_{CX(Y)} is the input impedance at terminals X (Y) of the OTA 306, dependent on the length (L) and width (W) of input differential pairs MOSFET of the OTA 306;

Further, g_{m, FBN(FEN)} equals N(M) times of the trans-conductance of an inverter in the FBN (FFN).

R_{o, FBN(FEN)} equals the reciprocal of N(M) multiplied the output impedance of an inverter in the FBN (FFN).

Also, X_{C, FBN(FEN)} equals the reciprocal of N(M) multiplied by the capacitive impedance of an inverter in the FBN (FFN). C_{Y} is the total capacitance at terminal Y.

To force α to be unity, R_{(o, FBN)} should be significantly large. In addition, β is a ratio of M to N if the inverters are perfectly matched.

As the X terminal senses a current signal, its impedance should be significantly small by increasing both the R_{o, FBN} and the open loop gain of the OTA, (gₘ*Rₒ ∥X_{Co}). Further, the Z terminal acts as a current source, its impedance should be large so, R_{o, FEN} should be designed large.

FIG. 5 illustrates an exemplary block diagram representation 500 of a capacitance multiplier including a cascaded connection of a plurality of M-CCII blocks, according to certain embodiments.

As shown in FIG. 5, the positive and negative capacitor multiplier is construed by the cascaded connection of M-CCII blocks 302-1, 302-2 . . . , 302-i.

The building block of this CM comprises a plurality of blocks of M-CCII (i.e., M-CII₁, M-CII₂, . . . , M-CIIᵢ). Each of the M-CCIIs 302-1, 302-2 . . . , 302-i comprises two input terminals (X, Y) and one output terminal (Z). For M-CCII₁, two input terminals are X₁, Y₁ and output terminal Z₁. For M-CCII₂, two input terminals are X₂, Y₂ and output terminal Z₂. Similarly, for M-CCIIᵢ, two input terminals are Xᵢ, Yᵢ and output terminal Zᵢ.

A grounded base capacitor C_{B} is connected to the X terminal of the M-CCII₁. Using the M-CCII's characteristics in equation (1), the equivalent capacitance (Ceq.) is given by equation (7) defined below.

$${C}_{eq .} = {( - \frac{M}{N} )}^{i} * {C}_{B} ; \qquad ( 7 )$$

The number of stages, i, can be odd or even to obtain a negative and a positive capacitance, respectively.

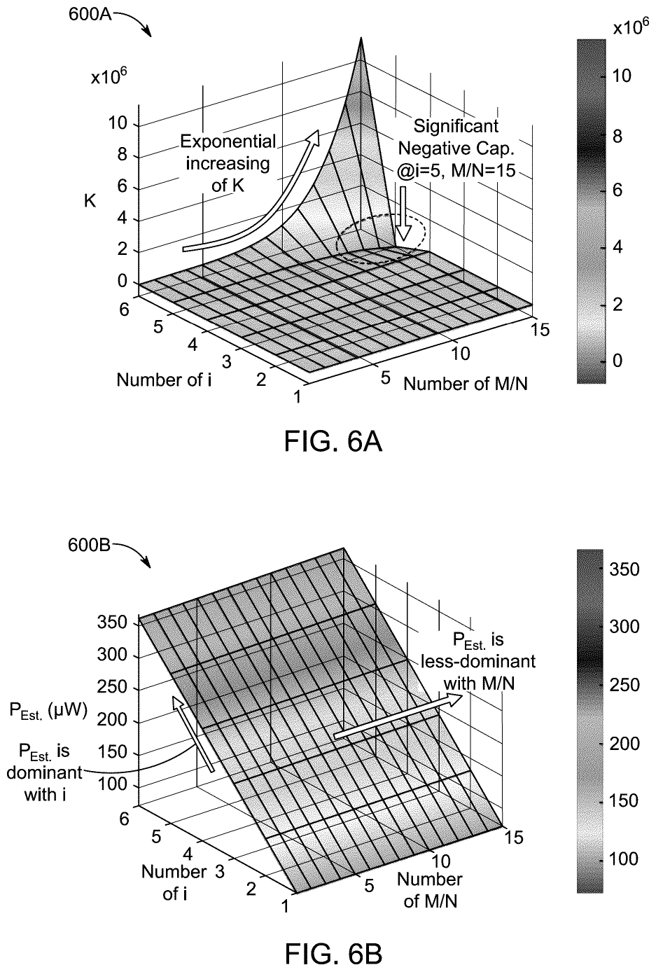

FIG. 6A illustrates a graph diagram 600A representing the multiplication factor with respect to the number of M/N and the number of i, according to certain embodiments.

The multiplication factor is given by K=(−M/N)ⁱ.

Given the M, N, and i values, static power consumption (P_{Est.}) can be estimated by equation (8) defined below:

$${P}_{Est} = {P}_{Bias} + ( {P}_{OTA} + ( M + N ) * {P}_{Inv} ) * i \qquad ( 8 )$$

As illustrated in FIG. 6A, the graph 600A is three-dimensional graph having an x-axis representing the number i, a y-axis representing the number M/N, and a z-axis representing the multiplication factor K.

The profile of the multiplication factor with respect to the values of M/N and i indicates that K increases exponentially with i and could be negative or positive.

FIG. 6B illustrates a graph diagram representing an estimated power consumption with respect to the number M/N and the number i, according to certain embodiments.

As illustrated in FIG. 6B, the graph 600B is three-dimensional graph having an x-axis representing the number i, a y-axis representing the number M/N, and a z-axis representing the estimated power consumption P_{Est}.

The dominant factor of the estimated power P_{Est} is the number of stages (i), while increasing M/N has a negligible effect on the estimated power. Thus, the trade-off between selecting M/N and i can be considered to compromise between a large K and power consumption.

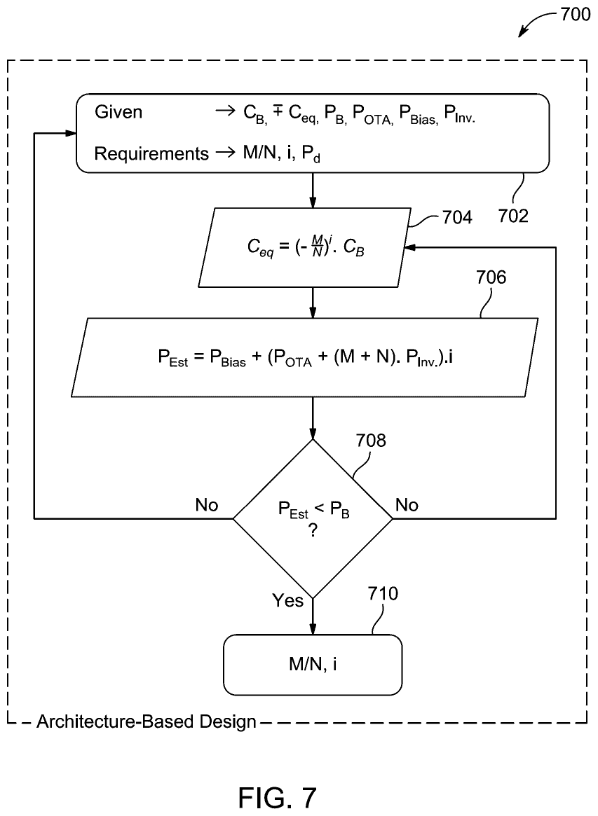

FIG. 7 illustrates an exemplary flow diagram 700 of an architecture-based design methodology, according to certain embodiments.

Based on the analysis outlined above, a design methodology is introduced to streamline the redesign process and accelerate analog design procedures. Additionally, it addresses the nonlinear relationship between component size and application specifications, leading to significant time and effort savings and reduced time to market.

A design methodology for the architecture (shown with reference to FIG. 5) is shown in FIG. 7 to facilitate the redesign of the architecture-based design methodology.

At step 702, target specifications and parameters are provided such as C_{B}, ∓C_{eq.}, budget power (P_{B}), P_{OTA}, P_{Bias}, and P_{Inv}. Then, a set of M/N and i can be determined according to the given C_{B} and ∓C_{eq.}.

At step 704, the value of C_{eq} is determined based on the values of M, N and i.

At step 706, the estimated power (P_{Est}) is calculated.

At step 708, the estimated power (P_{Est}) is compared to the budget power (P_{B}).

At step 710, upon detecting the estimated power (P_{Est}) is less than the budget power (P_{B}), the values of M/N and i are considered. Upon detecting the estimated power (P_{Est}) is greater than the budget power (P_{B}), the steps 702 and 704 are performed again.

In an example, when C_{B}=1 pF, C_{eq.}>50 nF, P_{B}≤300 μW, P_{OTA}≤60 μW, P_{Inv.}=0.06 μW, and P_{Bias}≤15 μW, M, N, and i can be selected as 15, 1, and 4, respectively, to attain the given specifications. Then, the expected power dissipation would be less than 250 μW.

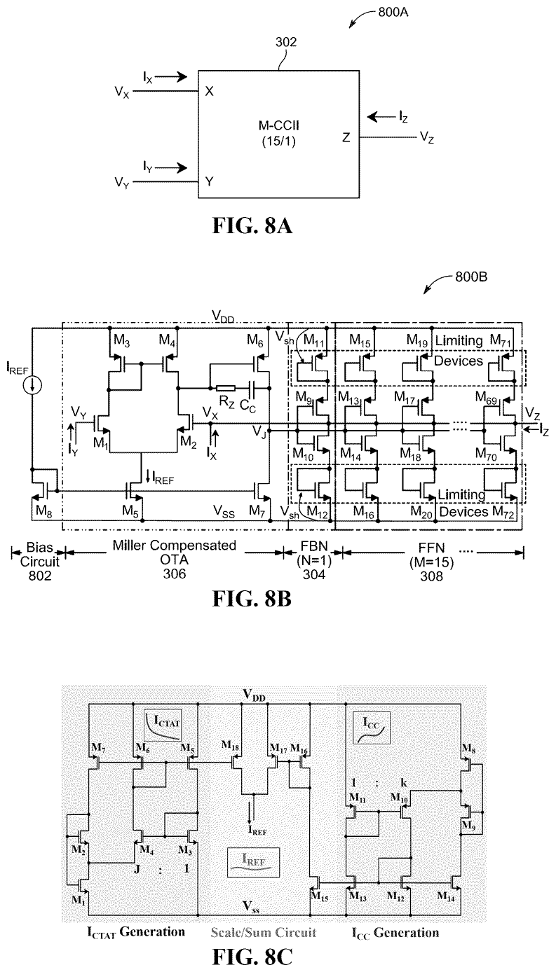

FIG. 8A illustrates an exemplary schematic diagram 800A of the M-CCII 302 (with M=15, N=1), according to certain embodiments.

The M-CCII 302 of FIG. 8A comprises two inputs (X, Y) and one output (Z). The input comprises voltages and currents V_{X}, I_{X} and V_{Y}, I_{Y}. The output comprises V_{Z} and I_{Z}.

FIG. 8B illustrates an exemplary circuit diagram 800B of the M-CCII 302 (with M=15, N=1), according to certain embodiments.

The circuit diagram of the M-CCII 302 comprises a bias circuit 802, a Miller-compensated operational transconductance amplifier (OTA) 306, the FBN 304 and the FFN 308.

In an embodiment, one or more CMOS structures may be used to realize the OTA 306 and CMOS inverter of the M-CCII 302 to enhance the accuracy of voltage and current gain and increase the output current capabilities while consuming low power. The Miller compensated OTA 306 drives large capacitive loads up to several pico-farads. The CMOS inverter is adapted to reduce the static current by adding NMOS and PMOS as limiting current devices. The bias circuit 802 may be used to provide the reference current (IREF).

The bias circuit 802 comprises NMOS differential input and a current source (I_{REF}).

For example, the bias circuit can include a separate circuit for providing the reference current (I_{REF}) to the OTA. FIG. 8C illustrates an exemplary stable current reference circuit based on a curvature compensation scheme. I_{REF} is a summation of a complementary to absolute temperature current (I_{CTAT}) and curvature-compensation current (I_{CC}) with a proper scaling to achieve high performance with process-voltage-temperature (PVT) variations.

The Miller compensated OTA 306 comprises a first stage and a second stage. The first stage consists of M₁ and M₂ representing the NMOS differential input pair with active load of M₃ and M₄. M₅ provides the tail current. The second stage, consisting of M₆ and M₇, acts as a common source amplifier. The two stages are biased by M₈, which provides the required reference current (I_{REF}) from the bias circuit 802. Resistor R_{Z} and capacitor C_{C} are used to improve the stability and phase margin of the OTA 306 while driving a significant capacitive load.

In one example, the reference current I_{REF} can be determined by the subsequent procedures:
To keep the phase margin of the Miller-compensated OTA larger than 65° to guarantee high stability, the value of the compensation capacitor (Cc) should be greater than 0.2 times the given capacitive load (C_{L}).
Then, I_{REF} equals the product of the targeted slew rate (SR) and the compensation capacitor (Cc), I_{REF}=SR*Cc.

The, the dimension of M₈ can be determined by the following procedures:
The dimension of M₅ is determined firstly,

$${( \frac{W}{L} )}_{5} = \frac{2 {I}_{R E F}}{{{β}_{n} ( {V}_{o d , 5} )}^{2}} ,$$

by given the overdrive voltage of M₅ (V_{od,5}) and I_{REF}.
Since M₈ and M₅ have been designed to include the same current level, the dimension of M₈ equals the dimension of M₅, i.e.,

$${( \frac{W}{L} )}_{5} = {( \frac{W}{L} )}_{8} .$$

Both components R_{Z} and C_{C} are essential components within the Miller-compensated OTA. Their values are dependent on the Miller-compensated OTA's operation. The value of C_{C} can be determined as follows:
To keep the phase margin of the Miller-compensated OTA larger than 65° to guarantee high stability, the value of the compensation capacitor (C_{C}) should be greater than 0.2 times the given capacitive load (C_{L}).

The value of R_{Z} should be greater than the reciprocal of the transconductance of M₆ (gₘ₆), i.e.,

$${R}_{Z} > \frac{1}{g m 6} .$$

Furthermore, it is tuned to improve the stability of the Miller-compensated OTA for wideband operation, because R_{Z} would function to cancel out the 2^{nd} pole and facilitate increasing the gain-bandwidth product.

In an embodiment, the FBN 304 may have a single modified CMOS inverter using M₉-M₁₂.

In an embodiment, the FFN 308 may have, without limiting the scope of the invention, fifteen modified CMOS inverters using M₁₃-M₇₂, including the limiting current devices.

In FIG. 8B, the limited devices for the FBN 304 and FFN 308 are surrounded by a dashed box. As depicted in FIG. 8B, the modified CMOS inverter in the FBN 304 includes M₉-M₁₂. The upper transistor (M₁₁) and lower transistor (M₁₂) act as the current-limiting devices. Furthermore, in the FFN 308, M₁₃-M₇₂ represent the 15 modified CMOS inverters, the upper transistors (M₁₅, M₁₉, . . . , and M₇₁) and the lower transistors (M₁₆, M₂₀, . . . , and M₇₂) present the limiting devices.

The grounded positive and negative capacitance multiplier 302 comprises a first number (i) of current conveyors connected in a cascaded manner. Each current conveyor includes the transconductance amplifier (OTA) 306, the feedback network (FBN) 304 including a second number (N) of CMOS inverters, and the feedforward network (FFN) 308 including a third number (M) of CMOS inverters. The OTA 306 is a Miller-compensated OTA. The Miller-compensated OTA 306 is further configured to receive a reference current from a bias circuit. The bias circuit 802 includes a first stage for generating a complementary to absolute temperature current, a second stage for generating a curvature-compensation current, and a third stage for applying a scaling factor to a summation of outputs of the first and second stages. The Miller-compensated OTA 306 includes a first stage configured as an NMOS differential input pair with active loads, and a second stage configured as a common source amplifier. Both the first and second stages are biased by the bias circuit 802. The reference current is provided from the bias circuit 802 through a CMOS transistor to the first and second stages of the Miller-compensated OTA 306. The Miller-compensated OTA 306 includes a compensation resistor and a compensation capacitor, and the phase margin of the Miller-compensated OTA 306 is configured to be larger than 65°. Each CMOS inverter of the second number (N) of CMOS inverters in the FBN 304 has a pair of NMOS and PMOS transistors as current-limiting devices. Each CMOS inverter of the third number (M) of CMOS inverters in the FFN 308 has a pair of NMOS and PMOS transistors as current-limiting devices.

The base capacitor is connected between a first input terminal of a first current conveyor of the cascaded current conveyors and a ground node. A multiplied capacitance is obtained between a second input terminal of the first current conveyor and the ground node, with a capacitance value given by

$${( - \frac{M}{N} )}^{i} * {C}_{B} ,$$

where C_{B} represents a capacitance of the base capacitor, i=4, M=15, and N=1. When i is an odd number, the multiplied capacitance has a negative value, and when i is an even number, the multiplied capacitance has a positive value.

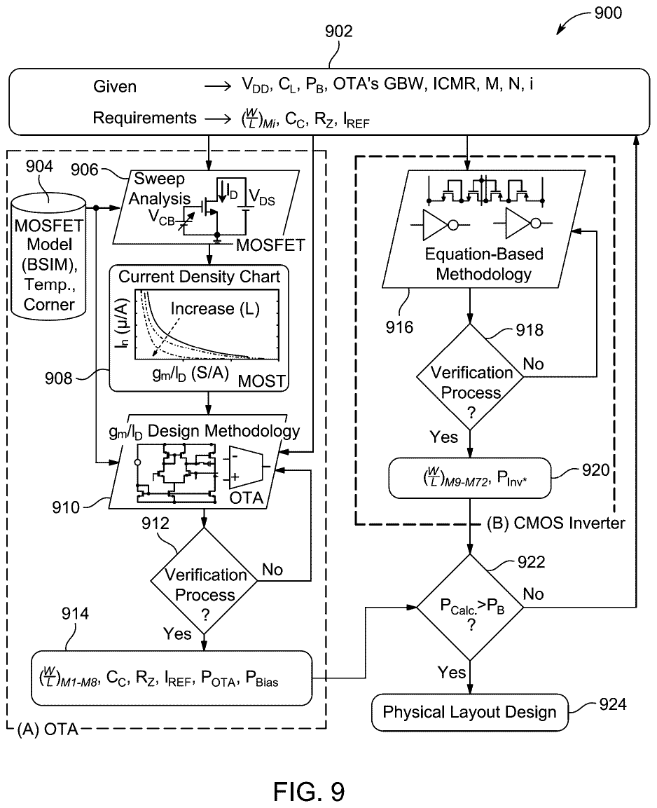

FIG. 9 illustrates an exemplary flow diagram 900 of a circuit-based design methodology, according to certain embodiments.

The circuit-based design methodology overcomes the nonlinear relationships between the transistor's sizing and the required specifications of the architecture-based design methodology.

At step 902, the requirements of the circuit-based design methodology are provided such as a supply voltage (VDD), a load capacitance (CL), a OTA's gain bandwidth product (GBW), an input common mode range (ICMR), PB, M, N, and i.

The circuit-based design methodology consists of two essential sections: OTA and CMOS inverter design procedures.

The OTA design procedure is explained with the steps 904-914:

At step 904, the MOSFET model, the Berkeley Short-channel IGFET Model (BSIM), is identified, and the temperature range and corner are set. The MOSFET model's temperature corner is then simulated.

In an aspect, the BSIM helps to predict the electrical characteristics of transistors under different operating conditions, such as voltage, temperature, and process variations, to ensure that circuit designs work properly in real-world scenarios.

At step 906, a sweep analysis of the output of the MOSFET model is performed to extract gₘ/I_{D} without the need for complex equations to provide current density charts. gₘ and I_{D} are notations for the transconductance and drain current of the MOSFET, respectively.

In an aspect, the sweep analysis of the MOSFET comprises step of varying one or more parameters (e.g., voltage or current) over a specific range and observing the corresponding changes in the MOSFET's behavior. The sweep analysis is useful for understanding the performance of a MOSFET under different operating conditions (e.g., varying gate voltage (V_{GS}), drain voltage (V_{DS}), or temperature).

At step 908, a current density chart is plotted. gₘ/I_{D} and normalized current (Iₙ=I_{D}/(W/L)) are referred to as the x-axis and y-axis, respectively, in the current density chart. The sweep analysis is recorded with different lengths, creating several densities of charts. Upon generating the chart density of the MOSFET, gₘ/I_{D} design methodology is utilized to get the dimensions of each transistor.

The current density chart for the MOSFET provides the relationship between the current (Iₙ) (the amount of current per unit area) and parameter gₘ/I_{D} .

At step 910, gₘ/I_{D} design methodology is performed for the OTA based on output of MOSFET model, the requirements of circuit-based design.

At step 912, a set of processes, such as DC, AC, and transient test benches, are executed to verify the functionality of the OTA with the bias circuit. Further, it is determined whether the verification process of the performed gₘ/I_{D} design methodology is successful.

At step 914, upon detecting the verification process of the performed gₘ/I_{D} design methodology is successful, the parameters (W/L)_{M1-M8}, C_{c}, R_{Z}, I_{REF}, P_{OTA}. P_{Bias} are determined.

Conversely, upon detecting the verification process of the performed gₘ/I_{D} design methodology is not successful, the gₘ/I_{D} design methodology step is repeated.

With respect to the CMOS invertor, a modified CMOS inverter is used to deliver output current. The maximum current of the modified CMOS inverter is given by:

$${I}_{Max} = \frac{μ * {C}_{o x} * W}{2 L} {( {V}_{J} - {V}_{s h} - {V}_{Th} )}^{2} \qquad ( 12 )$$

where μ is the carrier mobility, Cₒₓ is the gate capacitance of CMOS transistor, Vₛₕ is the shifting voltage corresponding to adding the limiting current device, and Vₜₕ is the transistor's threshold voltage.

The maximum current is reduced by using limiting current devices. The static consumed current is reduced by a large transistor's length. This leads to a reduction in speed.

The modified CMOS inverter reduces the static current by more than 156×. It is designed to optimize the used silicon area, power condition, and maximum delivered current (I_{Max}).

At step 918, a verification process is performed to check the functionality of the modified CMOS invertor over the conventional CMOS invertor.

At step 920, upon detecting the functionality of the modified CMOS invertor over the conventional CMOS invertor is satisfactory, the parameters (W/L)_{M9-M72}, P_{INV} are determined.

After sizing the OTA 306 with the bias circuit 802 and the modified CMOS inverter, the calculated power (P_{CALC.}) can be calculated.

At step 922, the calculated power (P_{CALC}) is compared against the budget power (P_{B}) to check whether P_{CALC}>P_{B}.

At step 924, upon detecting P_{CALC}>P_{B}, the physical layout design is finalized. Conversely, upon detecting the P_{CALC}<P_{B}, the requirements of the circuit-based design methodology are checked and the circuit-based design methodology is reperformed.

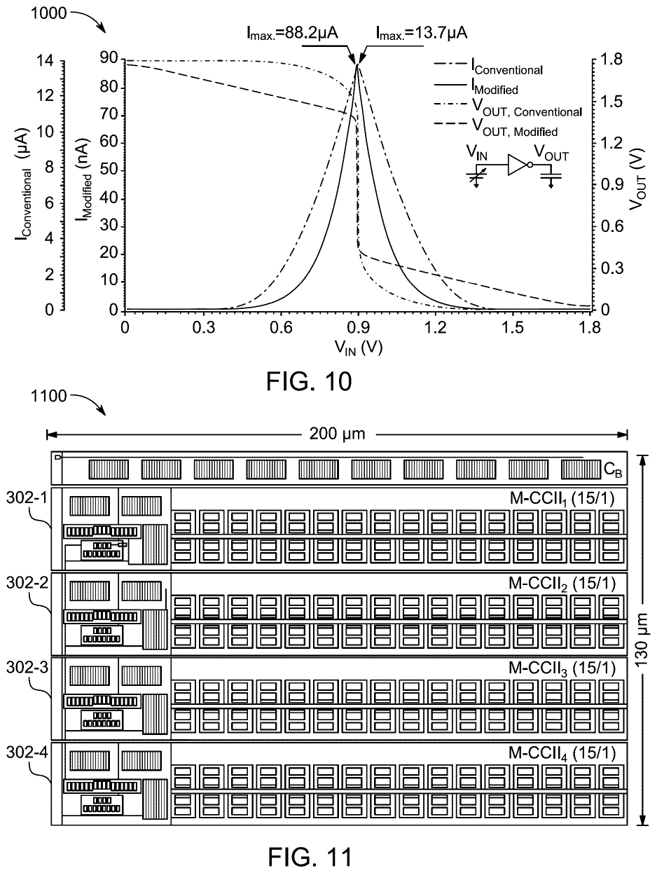

FIG. 10 illustrates a graph diagram 1000 representing the input-output transfer characteristics of the conventional and the modified CMOS inverters, according to certain embodiments.

As illustrated in FIG. 10, the input and the output of the conventional CMOS inverter comprise I_{conventional}, V_{OUT, Conventional}. The input and output of modified CMOS inverter comprise I_{Modified}, V_{OUT, Modified}.

The graph 1000 shows I_{Conventional} (uA) and I_{Modified} (nA), input voltage V_{IN} (V) and output voltage V_{OUT} (V). The graph 1000 also shows I_{max, Conventional} and I_{max, Modified}. In an example, I_{max, Conventional}=13.7 uA and I_{max, Modified}=88.2 uA.

FIG. 11 illustrates an exemplary schematic diagram 1100 of the M-CCII-based capacitor multiplier (CM), according to certain embodiments.

As illustrated in FIG. 11, the capacitor multiplier (CM) with four blocks of the M-CCII (with M=15 and N=1) is designed using technology (e.g., TSMC 0.18 μm CMOS technology) and a single power supply (VDD) of 1.8 V. The transistors' dimensions and design parameters are listed in Table 1 below.

TABLE 1
Transistors' dimensions for the proposed M-CCII

| IP block | Transistor | ^{b}# Fingers | W/L (μm/μm) |
|---|---|---|---|
| ^{a}OTA | M₁-M₂ | 2 | 2/1 |
| | M₃-M₄, M₅ | 2 | 4/1 |
| | M₆ | 12 | 24/1 |
| | M7 | 6 | 12/1 |
| Modified CMOS | M₉-M₇₂ | 1 | 2/5 |
| Inverter | | | |
| ^{c}Bias circuit | M₈ | 2 | 4/1 |

^{a}C_{C} = 0.2 pF, R_{Z} = 30 KΩ,
^{b}Number of fingers for interdigitating,
^{c}I_{REF} = 8 μA

As shown in FIG. 11, the schematic diagram of the M-CCII-based CM has been designed using layout editor tools. The schematic diagram occupies a silicon area of 200 μm×130 μm. The designed M-CCII-based CM is simulated. Procedures (e.g., design rule check (DRC), layout versus schematic (LVS), and parasitic extraction (PEX)) are carried out using caliber tools. For PEX, the distribution of resistor, capacitor, and coupling capacitor (R+C+CC) at a nominal temperature of 27° C. are set to extract the parasitic elements.

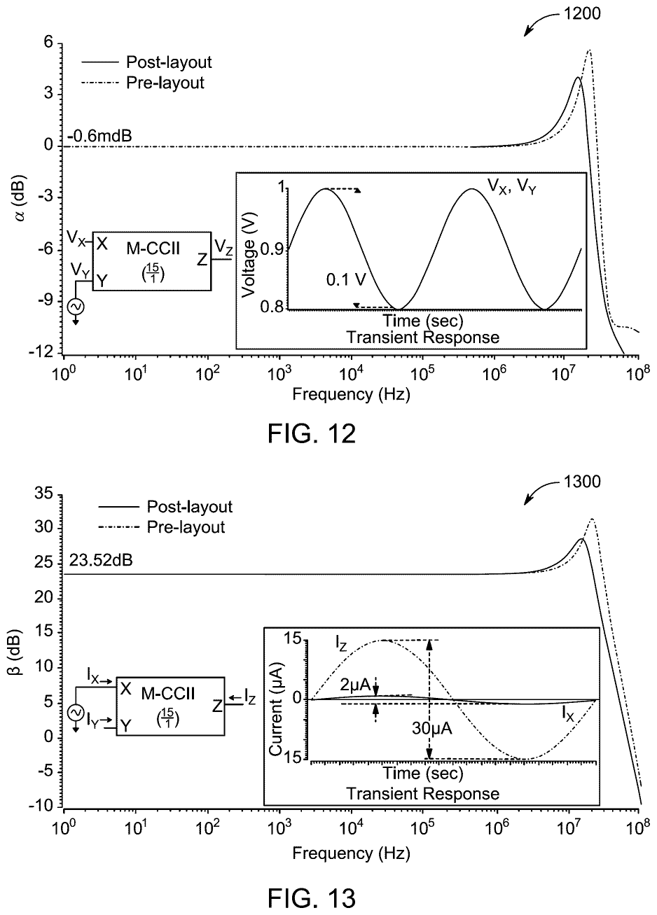

FIG. 12 illustrates a graph diagram 1200 representing a frequency response to a voltage gain, according to certain embodiments.

To examine the functionality performance of M-CCII (with M=15, N=1), the supply voltage, and temperature are set at typical-typical, 1.8 V and 27° C., respectively. The pre- and post-layout simulation results of the voltage gain (α) are shown in FIG. 12.

The x-axis of the graph 1200 represents frequency (Hz), and the y-axis represents voltage gain (α) dB.

The frequency response indicates that the bandwidth (α_{BW}) reaches 1.7 MHz with a DC gain of −0.6 m dB. In addition, the transient response shows that V_{X} tracks V_{Y} when applying a sin wave with an amplitude of 0.1 V and frequency of 10 kHz.

FIG. 13 illustrates a graph diagram 1300 representing a frequency response to a current gain, according to certain embodiments.

The pre- and post-layout simulation results of the current gain (β) dB.

The x-axis of the graph 1300 represents frequency (Hz), and the y-axis represents current gain (β) dB.

As shown FIG. 13, the frequency response of the current gain (β) has a bandwidth (β_{BW}) of 24.2 MHz with DC gain of 23.52 dB. In the transient response, the injected current I_{X} with an amplitude of 2 μA peak-peak and frequency of 10 kHz is amplified by 15 times as depicted. Upon applying a sinusoidal input peak current (I_{X}) of 100 nA at a frequency of 100 Hz, the total harmonic distortion (THD) was observed to be 1.86%.

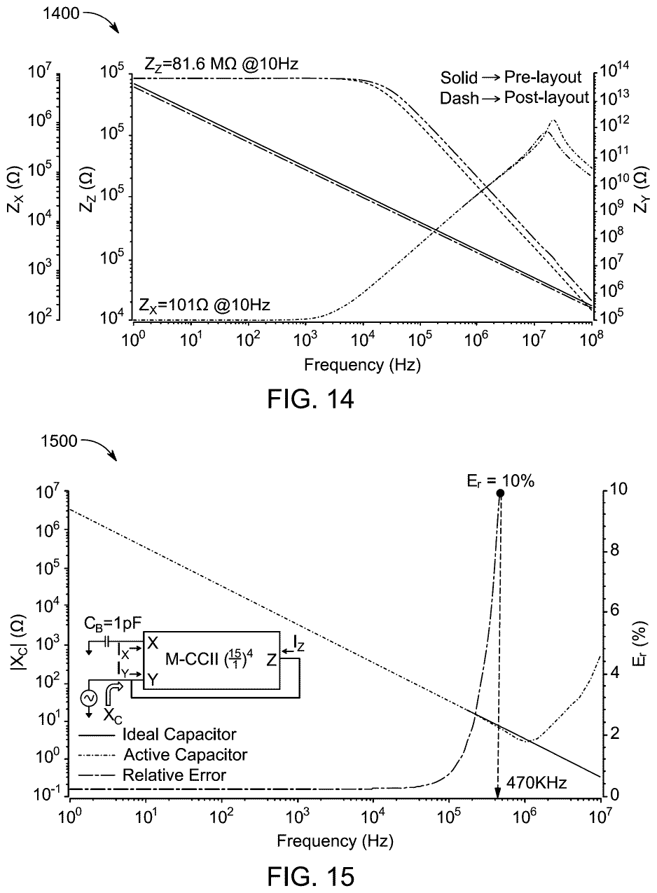

FIG. 14 illustrates a graph diagram 1400 representing impedances at X, Y, and Z terminals, according to certain embodiments.

The x axis of the graph 1400 represents frequency (Hz) and the y-axis represents Z_{X} (Ω), Z_{Y} (Ω), and Z_{Z} (Ω).

As shown in FIG. 14, the impedances at X, Y, and Z terminals of the M-CCII (with M=15, N=1) are presented for pre- and post-layout simulation. Z_{X} exhibits a low value at 101Ω at low frequency, enabling sensing of a low current signal effectively. However, Z_{Z} is at a high level at 81.6 MΩ, enabling pushing current to the load. Also, Z_{Y} exhibits an input capacitance of 4 fF.

FIG. 15 illustrates a graph diagram 1500 representing impedance of the M-CII-based CM, according to certain embodiments.

The graph shows the behavior of an ideal capacitor, an active capacitor and relative error over the frequency.

As shown in FIG. 15, the impedance of the capacitor multiplier is based on M-CCII blocks and a base capacitor of 1 pF, where the base capacitor is an ideal capacitor, with M=15, N=1, and i=4. The equivalent capacitance follows the ideal capacitor till 470 kHz with a corresponding relative error of 10%.

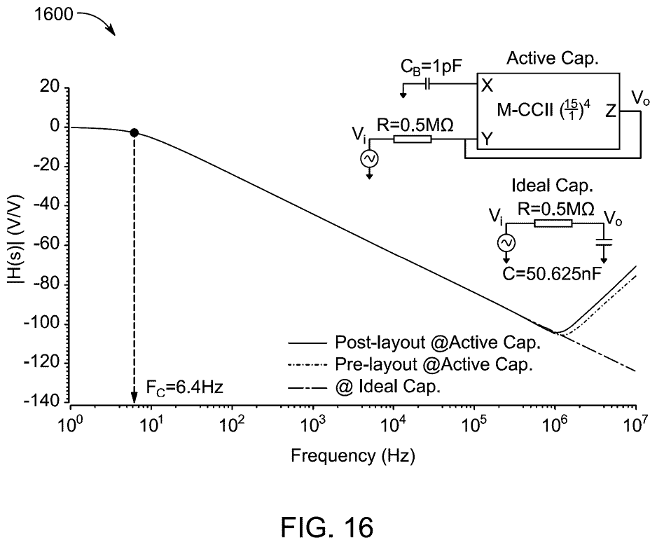

FIG. 16 illustrates a graph diagram 1600 representing a frequency resonance of the low-pass-filter (LPF), according to certain embodiments.

As illustrated in FIG. 16, the x-axis of the graph 1600 shows frequency resonance |H(s)|(V/V) of the low pass filter and the y-axis represents frequency (Hz).

The frequency resonance |H(s)| is derived for the low pass filter with the proposed CM. The simulation outcomes for the LPF with the CM and ideal capacitor align very well. The low-pass filter includes a resistor of 0.5 MΩ. The capacitance multiplier has a multiplication factor of 50,625; the base capacitor has a capacitance of 1 pF. As a result, the low-pass filter has a corner frequency of 6.4 Hz.

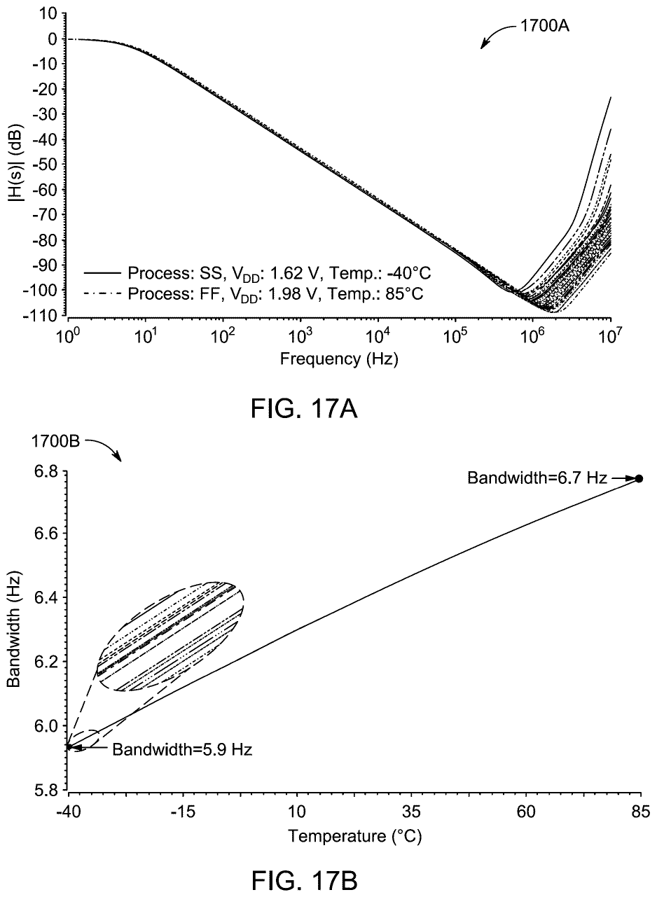

FIG. 17A illustrates a graph diagram 1700A representing the frequency response of the LPF including the M-CCII-based capacitor multiplier, according to certain embodiments.

To assess process-voltage-temperature (PVT) variations, the frequency responses of the M-CCII and the low-pass-filter at five corners {typical-typical (TT), fast-fast (FF), slow-slow (SS), fast-slow (FS), slow-fast (SF)}, three levels of power supply {±10% of nominal V_{DD}} from 1.62 V to 1.98 V, and a temperature range from −40° C. to 85° C. are executed.

As illustrated in FIG. 17A, the x-axis of the graph 1700A shows the frequency resonance |H(s)| (dB) of the low pass filter and the y-axis represents frequency (Hz). The graph 1700A shows the frequency response of the LPF including the M-CCII-based capacitor multiplier with the five corners (TT, FF, SS, FS, SF). Table 4 below reports the worst case of the M-CCII's parameters, according to the pre-layout simulation results.

TABLE 4
Process-Voltage-Temperature Results of the M-CCII (15/1)

| | | | Conditions | | | |
|---|---|---|---|---|---|---|
| Parameter | | Value | Process | Voltage | Temperature | |
| ¹\|α\| | Min | 605.83 | TT | 1.98 V | −40° | C. |
| (μdB) | Max | 1085.1 | SF | 1.62 V | 85° | C. |
| α_{BW} | Min | 0.302 | SS | 1.62 V | −40° | C. |
| (MHz) | Max | 9.694 | FF | 1.98 V | 85° | C. |
| ¹\|β\| | Min | 23.5218 | FF | 1.98 V | −40° | C. |
| (dB) | Max | 23.5219 | SS | 1.62 V | 85° | C. |
| β_{BW} | Min | 10.518 | SS | 1.62 V | −40° | C. |
| (MHz) | Max | 43.465 | FF | 1.98 V | −40° | C. |
| ¹Z_{X} | Min | 31.421 | TT | 1.98 V | 85° | C. |
| (Ω) | Max | 2414.2 | SS | 1.62 V | −40° | C. |
| ¹Z_{Z} | Min | 13.6 | FF | 1.98 V | 85° | C. |
| (MΩ) | Max | 1931.5 | SS | 1.62 V | −40° | C. |
| C_{Y} | Min | 3.926 | SS | 1.98 V | 85° | C. |
| (fF) | Max | 4.689 | FF | 1.62 V | −40° | C. |

¹@Frequency = 10 Hz

FIG. 17B illustrates a graph diagram 1700B representing the corresponding bandwidth of the LPF according to PVT variations, according to certain embodiment.

As illustrated in FIG. 17B, the x-axis of the graph 1700B shows temperature (° C.) of the low pass filter and the y-axis represents bandwidth (Hz). Table 3 reports the worst case of the LPF's bandwidth, according to pre-layout simulation results.

TABLE 3
Process-Voltage-Temperature Results of the LPF

Conditions

| Parameter | | Value | Process | Voltage | Temperature | |
|---|---|---|---|---|---|---|
| BW | Min | 5.9 | SS | 1.62 V | −40° | C. |
| (Hz) | Max | 6.7 | FF | 1.98 V | 85° | C. |

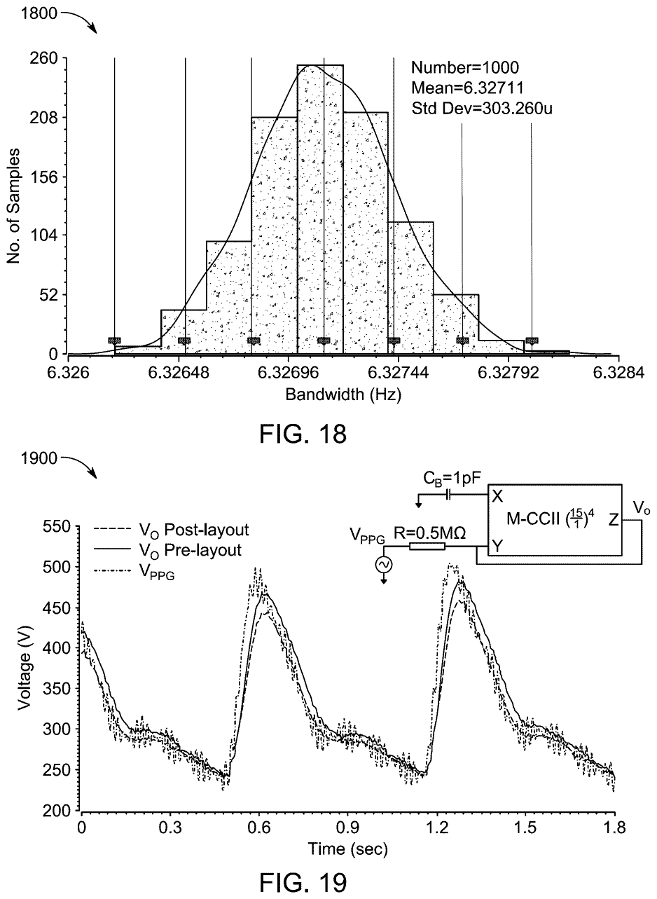

FIG. 18 illustrates a graph diagram 1800 representing Monte Carlo (MC) simulation results of the LPF's bandwidth, according to certain embodiments.

As illustrated in FIG. 18, the x-axis of the graph 1800 shows bandwidth (Hz) of the low pass filter and the y-axis represents the number of samples.

Monte-Carlo (MC) simulation at 27° C. and 1.8 V voltage supply was performed to evaluate the variability in performance caused by both process and mismatching. For 1000 runs of MC, a target yield is ±3σ. Utilizing a statistical distribution model, the MC setup randomly selects all the parameters of NMOS and PMOS transistors for each simulation run to assess mismatches and process modifications.

Herein, the coefficient of variance (C=(σ/μ)*100) can be exploited to indicate the variance between both the mean value (μ) and the standard deviation (σ). Tables 5 and 6 reports μ, σ, and CV of the proposed M-CCII's parameters and the LPF, respectively, also compared with the prior arts. CV of the proposed circuits are significantly low and indicates a well performance. Furthermore, Monte-Carlo results of the LPF's bandwidth is presented, including the mean value and the standard deviation, as shown in FIG. 18.

TABLE 5
Monte-Carlo Process and Mismatch Results of the M-CCII (15/1)

| Ref. | Run | Parameter | μ | σ | CV [%] |
|---|---|---|---|---|---|
| CM based | 1000 | \|α\| (mdB) | 0.731 | 0.083 | 11.354 |
| on | | α_{BW} (MHz) | 2.86837 | 0.036 | 1.2550 |
| CCII | | \|β\| (dB) | 23.5218 | 0.763μ | 3.2E−06 |
| | | β_{BW}(MHz) | 32.6 | 1.55 | 4.754 |
| | | Z_{X}(Ω) | 115.731 | 23.3 | 20.13 |
| | | Z_{Z}(MΩ) | 81.71 | 14.6 | 17.86 |
| | | C_{Y} (fF) | 4.0005 | 0.046 | 1.149 |

TABLE 6
Monte-Carlo Process and Mismatch Results of the LPF

| | | μ | σ | CV |
|---|---|---|---|---|
| Ref. | #run | [Hz] | [Hz] | [%] |
| CM based on CCII | 1000 | 6.32 | 0.3m | 0.05 |
| Ref-16 | 100 | 2.04K | 65 | 3.1 |
| Ref-5 | 100 | 23.53K | 3.6K | 15.3 |
| Ref-17 | 100 | 2.7K | 200 | 7.4 |
| Ref-18 | 100 | 11.4K | 276 | 2.4 |

FIG. 19 illustrates a graph diagram 1900 representing a noisy PPG signal and the LPF's output, according to certain embodiments. As illustrated in FIG. 19, the x-axis of the graph 1900 represents time (sec) and the y-axis represents voltage (V).

The design implements the LPF with FC=6.4 Hz to act as a block of the PPG sensor circuit, as shown in FIG. 1B. The noisy PPG signal due to 50-60 Hz power line interference can be filtered as shown in FIG. 19, which proves the functionality of the design. In contrast to the noisy signal, the output of the LPFs, whether pre- or post-layout, is much clearer and cleaner.

Table 7 presents the performance of the capacitor multiplier circuit based on the M-CCII and a comparison with the prior arts.

TABLE 7
Performance of the CM based on M-CCIIs and comparison with other systems

| | Building | Tech | SV | C_{B} | | | | | BW | PD | |
|---|---|---|---|---|---|---|---|---|---|---|---|
| References | Block | [nm] | [V] | [pF] | K | #NT | #AD | #PR | [MHz] | [μW] | ^{a}FOM₁ |
| CM | CCII | 180 | 1 · 8 | 1 (G) | 50625 | 285 | 4 | 0 | 0.47 | 250 | 19.736 |
| based | | | | | | | | | | | |
| on | | | | | | | | | | | |
| CCIIs | | | | | | | | | | | |
| Ref-13 | CCII, | 180 | 1 · 8 | 3 (F) | 3600 | 76 | 4 | 0 | 0.005 | 2.03 | 2.105 |
| | OTA | | | | | | | | | | |
| Ref-16 | MOV | 180 | 1 · 8 | 1 (F) | 750 | 24 | 1 | 0 | 0.1 | 420 | 8.6806 |
| | DTA | | | | | | | | | | |
| Ref-5 | CCC | 180 | 1 · 8 | 10 (F) | 150 | 35 | 1 | 0 | 10 | 600 | 0.119 |
| | DTA | | | | | | | | | | |
| Ref-6 | CFOA | 130 | 1 · 2 | 100 (G) | 500.5 | 29 | 1 | 2 (F), | 80 | 1600 | 0.0144 |
| | | | | | | | | 2 (G) | | | |
| Ref-4 | VDTA | 180 | 1 · 8 | 100 (F) | 20 | 4 | 1 | 1 (G) | 100 | 890 | 0.0069 |
| Ref-7 | VCII | 350 | 3 · 3 | 100 (F) | 50 | 34 | 2 | 1 (F), | 10 | 1500 | 0.0005 |
| Ref-8 | CFTA | 130 | 1 · 5 | 10 (F) | 10 | 54 | 2 | 2 (G) | 0.2 | 100 | 0.0014 |
| Ref-14 | DTMOS | 180 | 1 · 8 | 1 (F) | 6000 | 32 | 2 | 0 | 0.001 | 0.814 | 34.7222 |
| Ref-17 | CDTA | 180 | 1 · 8 | 1 (F) | 300 | 31 | 1 | 0 | 8 | 720 | 2.6882 |
| Ref-1 | ICFOA | 1.8 | 2 · 5 | 50 (F) | 318 | 46 | 1 | 1 (F), | 0.03 | 24 | 0.0092 |
| | | | | | | | | 1 (G) | | | |
| Ref-18 | VDTA | 180 | 1 · 8 | 1 (F) | 150 | 1 | 1 | 0 | 0.2 | 37 | 2.3148 |
| Ref-2 | VCII | 180 | 0 · 6 | 100 (F) | 100 | 12 | 1 | 0 | 0.04 | 0.09 | 0.069 |
| Ref-3 | CFOA | 180 | 1 · 5 | 20 (G) | 51 | 22 | 1 | 2 (F) | 6 | 100 | 0.012 |
| Ref-12 | D-OTA | 350 | 2 · 2 | 25 (F) | 28 | 37 | 2 | 0 | 0.008 | 1320 | 0.0001 |
| Ref-15 | D-OTA | 180 | 1 · 8 | 1 (F) | 6000 | 20 | 2 | 0 | 0.3 | 340 | 18.5 |
| Ref-9 | OTA | 500 | 2 · 7 | 18 (F) | 10.1 | 20 | 1 | 0 | 1 | 182000 | 0.005 |

| | | | | ^{e}Error | Silicon | |
|---|---|---|---|---|---|---|
| References | ^{b}FOM₂ | ^{c}FOM₃ | ^{d}FOM₄ | [%] | Area [mm²] | C_{eq-} |
| CM | 95.17 | 13.21 | 19.03 | 2.3 | 0.023 | B |
| based | | | | | | |
| on | | | | | | |
| CCIIs | | | | | | |
| Ref-13 | 8.86 | 1.47 | 1.77 | 8.6 | N/A | P |
| Ref-16 | 0.178 | 0.099 | 0.089 | N/A | 0.0008 | P |
| Ref-5 | 2.5 | 1.388 | 1.25 | N/A | 0.014 | P |
| Ref-6 | 2.50 | 2.08 | 0.25 | N/A | N/A | N |
| Ref-4 | 2.247 | 1.2484 | 0.561 | 4.3 | 0.001 | N |
| Ref-7 | 0.3333 | 0.0505 | 0.037 | 7.8 | N/A | B |
| Ref-8 | 0.02 | 0.0067 | 0.0022 | N/A | N/A | B |
| Ref-14 | 7.37 | 2.047 | 2.45 | N/A | 0.052 | P |
| Ref-17 | 3.333 | 1.852 | 1.666 | N/A | N/A | P |
| Ref-1 | 0.3975 | 0.159 | 0.0662 | N/A | V/A | P |
| Ref-18 | 0.81 | 0.45 | 0.405 | N/A | 0.0012 | P |
| Ref-2 | 41.66 | 69.44 | 20.83 | 2.5 | N/A | P |
| Ref-3 | 3.06 | 2.04 | 0.51 | N/A | N/A | P |
| Ref-12 | 3E−05 | 5E−05 | 0.0001 | N/A | 0.086 | P |
| Ref-15 | 5.294 | 1.4705 | 1.764 | N/A | 0.0038 | P |
| Ref-9 | 5E−05 | 2E−05 | 2E−05 | N/A | 0.25 | P |

${a}^{} {FOM}_{1} = \frac{K}{SV [ V ] * NT * ( AD + 1 ) * ( PR + 1 ) * {C}_{B} [ pF ]} ,$ ${b}^{} {FOM}_{2} = \frac{K * B W [ MHz ]}{{P}_{D} [ μ W ]} ,$ ${c}^{} {FOM}_{3} = \frac{B W [ MHz ]}{SV [ V ] * {P}_{D} [ μW ] * AD} ,$ ${d}^{} {FOM}_{4} = \frac{B W [ MHz ]}{{P}_{D} [ μW ] * ( AD + 1 ) * ( P R + 1 )} ,$ ${e}^{} Error \vert \frac{{C}_{eq . , ∼} .}{{C}_{eq . , Cacl .} - {C}_{eq . , ∼ .}} \vert ,$ G: Grounded,
F: Floating,
P: Positive,
N: Negative,
B: Both

As shown in FIG. 19, an extremely large multiplication factor of 50,625 is achieved with a relative error of 2.3% while consuming power of 250 μW with a single supply voltage of 1.8 V. Hence, a RC-low pass filter for filtering biosignals, such as PPG, from interferences can be realized when using a resistor of 0.5 MΩ and a base capacitor C_{B} of 1 pF. A series connection of a set of M-CCII blocks is used in the proposed configuration. A positive and negative equivalent capacitor can be obtained.

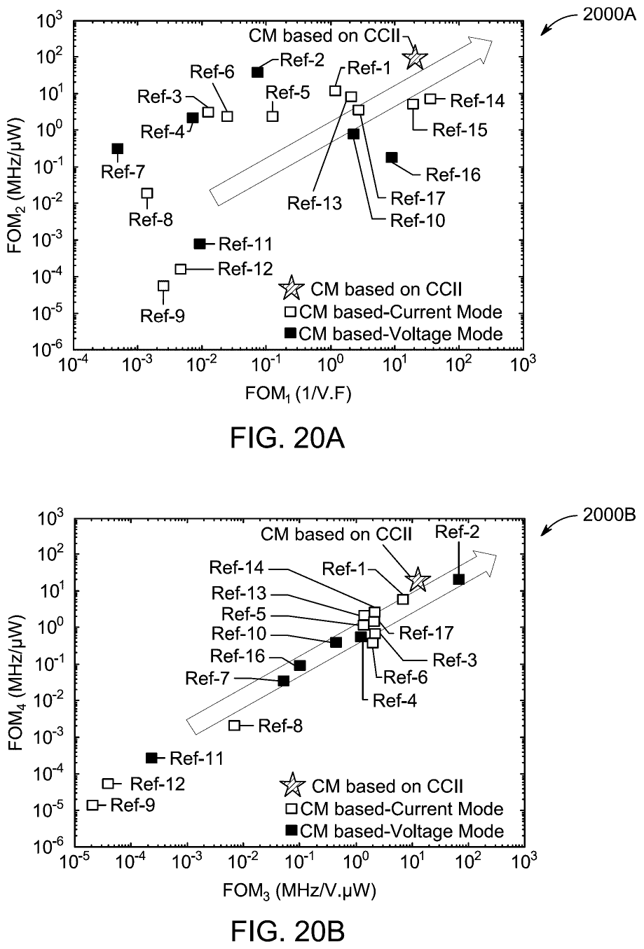

FIGS. 20A and 20B illustrate graph diagrams 2000A, 2000B representing a comparison of the CCII-based CM of the disclosure and multiple prior arts in terms of FOM₁ VS. FOM₂ and FOM₃ VS. FOM₄, respectively. As illustrated in FIG. 20A, the x-axis of the graph 2000A represents FOM₁ (1/V·F) and the y-axis represents FOM₂ (MHz/μW). As illustrated in FIG. 20B, the x-axis of the graph 2000B represents FOM₃ (MHz/V·μW) and the y-axis represents FOM₄ (MHz/μW).

Relevant metrics are included in FOM₁, FOM₂, FOM₃, and FOM₄ to assess the overall performance. Based on the FOMs displayed in FIGS. 20A and 20B, the CM circuit is found to be superior to prior efforts due to having an extreme multiplication factor compared to even the voltage mode active building blocks such as multiple output-voltage difference transconductance amplifier (MO-VDTA), voltage differencing transconductance amplifier (VDTA), and second-generation voltage conveyor (VCII).

The superiority of the CM is due to the dependence on a proposed accurate M-CCII. Only, 0.7 mdB and 25 μdB, respectively, are the deviations in voltage gain and current gain. The simulation results of the M-CCII are summarized. The M-CCII provides a significant FOM, which assesses the total performance of the M-CCII circuit, including the product of the voltage gain-bandwidth product (|α|*α_BW) and the current gain-bandwidth product (|β|*β_BW), then divided by the power consumption (PD).

A grounded positive and negative capacitor multiplier based on the M-CCII is designed and simulated using TSMC 180 nm CMOS technology. The CM achieves an extreme multiplication factor of 50,625 with a maximum error of 2.3%, utilizing a base capacitor of 1 pF. As a result, a low-pass filter with a corner frequency of 6.4 Hz is used to cancel out the 50-60 Hz power line and internal signal interference in the PPG sensor front-end circuit. The CM consumes a low power of 250 μW with a single supply voltage of 1.8 V and occupies a silicon area of 0.023 mm2. Monte Carlo and PVT variations have been used to assure the CM's proper performance.

First embodiment is illustrated with respect to FIGS. 3B, 4, 5, and 8B. The first embodiment describes a grounded positive and negative capacitance multiplier, comprising a first number (i) of current conveyors connected in a cascaded manner. Each current conveyor includes a transconductance amplifier (OTA), a feedback network (FBN) including a second number (N) of CMOS inverters, and a feedforward network (FFN) including a third number (M) of CMOS inverters. A base capacitor is connected between a first input terminal of a first current conveyor of the cascaded current conveyors and a ground node. A multiplied capacitance is obtained between a second input terminal of the first current conveyor and the ground node, with a capacitance value given by

$${( - \frac{M}{N} )}^{i} * {C}_{B} .$$

C_{B} represents a capacitance of the base capacitor. When i is an odd number, the multiplied capacitance has a negative value, and when i is an even number, the multiplied capacitance has a positive value.

In an aspect, the OTA is a Miller-compensated OTA.

In an aspect, the Miller-compensated OTA is further configured to receive a reference current from a bias circuit.

In an aspect, the bias circuit includes a first stage for generating a complementary to absolute temperature current, a second stage for generating a curvature-compensation current, and a third stage for applying a scaling factor to a summation of outputs of the first and second stages.

In an aspect, the Miller-compensated OTA includes a first stage configured as an NMOS differential input pair with active loads and a second stage configured as a common source amplifier. Both the first stage and the second stage are biased by the bias circuit.

In an aspect, the reference current is provided from the bias circuit through a CMOS transistor to the first and second stages of the Miller-compensated OTA.

In an aspect, the Miller-compensated OTA includes a compensation resistor and a compensation capacitor, and a phase margin of the Miller-compensated OTA is configured to be larger than 65°.

In an aspect, each CMOS inverter of the second number (N) of CMOS inverters in the FBN has a pair of NMOS and PMOS transistors as current-limiting devices.

In an aspect, each CMOS inverter of the third number (M) of CMOS inverters in the FEN has a pair of NMOS and PMOS transistors as current-limiting devices.

In an aspect, i=4, M=15, and N=1.

Second embodiment is illustrated with respect to FIGS. 1B, 3b, 4, 5, and 8B. The second embodiment describes a low-pass filter used for processing human biosignals. A multiplied capacitance obtained from a base capacitor and a grounded positive and negative capacitance multiplier, the capacitance multiplier including a first number (i) of current conveyors connected in a cascaded manner. Each current conveyor includes a transconductance amplifier (OTA), a feedback network (FBN) including a second number (N) of CMOS inverters, and a feedforward network (FFN) including a third number (M) of CMOS inverters. The base capacitor is connected between a first input terminal of a first current conveyor of the cascaded current conveyors and a ground node. The multiplied capacitance is obtained between a second input terminal of the first current conveyor and the ground node, with a capacitance value given by

$${( - \frac{M}{N} )}^{i} * {C}_{B} ,$$

where C_{B} represents a capacitance of the base capacitor. When i is an odd number, the multiplied capacitance has a negative value, and when i is an even number, the multiplied capacitance has a positive value.

In an aspect, the OTA is a Miller-compensated OTA.

In an aspect, the Miller-compensated OTA is further configured to receive a reference current from a bias circuit.

In an aspect, the bias circuit includes a first stage for generating a complementary to absolute temperature current, a second stage for generating a curvature-compensation current, and a third stage for applying a scaling factor to a summation of outputs of the first and second stages.

In an aspect, the Miller-compensated OTA includes a first stage configured as an NMOS differential input pair with active loads, and a second stage configured as a common source amplifier. Both the first stage and the second stage are biased by the bias circuit.

In an aspect, the reference current is provided from the bias circuit through a CMOS transistor to the first and second stages of the Miller-compensated OTA.

In an aspect, the Miller-compensated OTA includes a compensation resistor and a compensation capacitor, and a phase margin of the Miller-compensated OTA is configured to be larger than 65°.

In an aspect, each CMOS inverter of the second number (N) of CMOS inverters in the FBN has a pair of NMOS and PMOS transistors as current-limiting devices.

In an aspect, CMOS inverter of the third number (M) of CMOS inverters in the FFN has a pair of NMOS and PMOS transistors as current-limiting devices.

In an aspect, the low-pass filter further includes a resistor of 0.5 MΩ, the base capacitor has a capacitance of 1 pF, the capacitance multiplier has a multiplication factor of 50,625, and the low-pass filter has a corner frequency of 6.4 Hz.

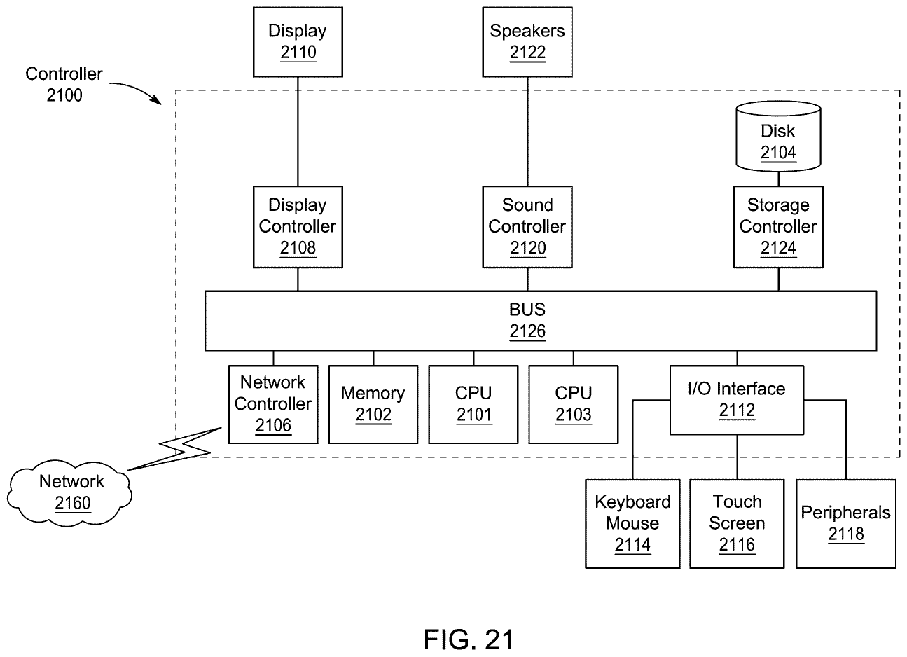

Next, further details of the hardware description of the computing environment of FIGS. 7 and 9 according to exemplary embodiments is described with reference to FIG. 21.

FIG. 21 shows an illustration of a non-limiting example of details of computing hardware, according to certain embodiments, for performing the functions of the exemplary embodiments.

In FIG. 21, a controller 2100 is described which includes a CPU 2101 which performs the processes described above/below. The process data and instructions may be stored in memory 2102. These processes and instructions may also be stored on a storage medium disk 2104 such as a hard drive (HDD) or portable storage medium or may be stored remotely.

Further, the present disclosure is not limited by the form of the computer-readable media on which the instructions of the inventive process are stored. For example, the instructions may be stored on CDs, DVDs, in FLASH memory, RAM, ROM, PROM, EPROM, EEPROM, hard disk or any other information processing device with which the computing device communicates, such as a server or computer.

Further, the present disclosure may be provided as a utility application, background daemon, or component of an operating system, or combination thereof, executing in conjunction with CPU 2101, 2103 and an operating system such as Microsoft Windows 7, Microsoft Windows 10, UNIX, LINUX, Apple MAC-OS and other systems known to those skilled in the art.

The hardware elements in order to achieve the computing device may be realized by various circuitry elements, known to those skilled in the art. For example, CPU 2101 or CPU 2103 may be a Xenon or Core processor from Intel of America or an Opteron processor from AMD of America, or may be other processor types that would be recognized by one of ordinary skill in the art. Alternatively, the CPU 2101, 2103 may be implemented on an FPGA, ASIC, PLD or using discrete logic circuits, as one of ordinary skill in the art would recognize. Further, CPU 2101, 2103 may be implemented as multiple processors cooperatively working in parallel to perform the instructions of the inventive processes described above.

The computing device in FIG. 21 also includes a network controller 2106, such as an Intel Ethernet PRO network interface card from Intel Corporation of America, for interfacing with network 2160. As can be appreciated, the network 2160 can be a public network, such as the Internet, or a private network such as an LAN or WAN network, or any combination thereof and can also include PSTN or ISDN sub-networks. The network 2160 can also be wired, such as an Ethernet network, or can be wireless such as a cellular network including EDGE, 3G, 4G, and 5G wireless cellular systems. The wireless network can also be WiFi, Bluetooth, or any other wireless form of communication that is known.

The computing device further includes a display controller 2108, such as a NVIDIA GeForce GTX or Quadro graphics adaptor from NVIDIA Corporation of America for interfacing with display 2110, such as a Hewlett Packard HPL2445w LCD monitor. A general purpose I/O interface 2112 interfaces with a keyboard and/or mouse 2114 as well as a touch screen panel 2116 on or separate from display 2110. General purpose I/O interface also connects to a variety of peripherals 2118 including printers and scanners, such as an OfficeJet or DeskJet from Hewlett Packard.

A sound controller 2120 is also provided in the computing device such as Sound Blaster X-Fi Titanium from Creative, to interface with speakers/microphone 2122 thereby providing sounds and/or music.

The general purpose storage controller 2124 connects the storage medium disk 2104 with communication bus 2126, which may be an ISA, EISA, VESA, PCI, or similar, for interconnecting all of the components of the computing device. A description of the general features and functionality of the display 2110, keyboard and/or mouse 2114, as well as the display controller 2108, storage controller 2124, network controller 2106, sound controller 2120, and general purpose I/O interface 2112 is omitted herein for brevity as these features are known.

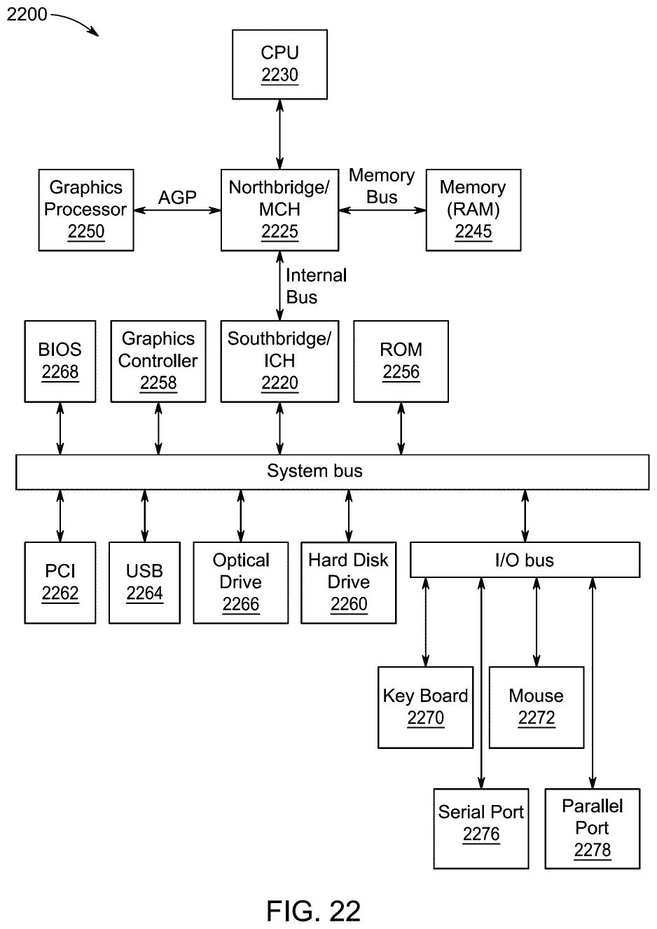

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown on FIG. 22.

FIG. 22 shows a schematic diagram of a data processing system, according to certain embodiments, for performing the functions of the exemplary embodiments. The data processing system is an example of a computer in which code or instructions implementing the processes of the illustrative embodiments may be located.

In FIG. 22, data processing system 2200 employs a hub architecture including a north bridge and memory controller hub (NB/MCH) 2225 and a south bridge and input/output (I/O) controller hub (SB/ICH) 2220. The central processing unit (CPU) 2130 is connected to NB/MCH 2225. The NB/MCH 2225 also connects to the memory 2245 via a memory bus, and connects to the graphics processor 2250 via an accelerated graphics port (AGP). The NB/MCH 2225 also connects to the SB/ICH 2220 via an internal bus (e.g., a unified media interface or a direct media interface). The CPU Processing unit 2230 may contain one or more processors and even may be implemented using one or more heterogeneous processor systems.

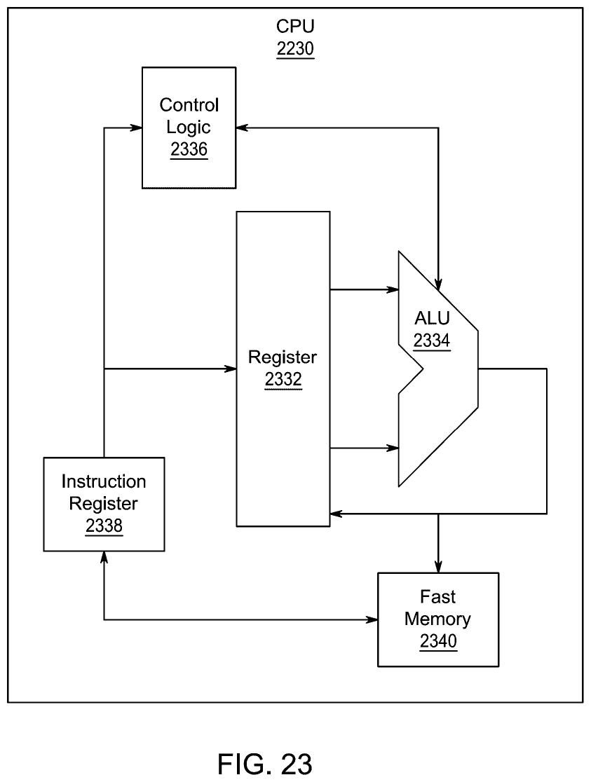

For example, FIG. 23 shows one implementation of CPU 2130. In one implementation, the instruction register 2338 retrieves instructions from the fast memory 2340. At least part of these instructions are fetched from the instruction register 2338 by the control logic 2336 and interpreted according to the instruction set architecture of the CPU 2130. Part of the instructions can also be directed to the register 2332. In one implementation the instructions are decoded according to a hardwired method, and in another implementation the instructions are decoded according to a microprogram that translates instructions into sets of CPU configuration signals that are applied sequentially over multiple clock pulses. After fetching and decoding the instructions, the instructions are executed using the arithmetic logic unit (ALU) 2334 that loads values from the register 2332 and performs logical and mathematical operations on the loaded values according to the instructions. The results from these operations can be feedback into the register and/or stored in the fast memory 2340. According to certain implementations, the instruction set architecture of the CPU 2130 can use a reduced instruction set architecture, a complex instruction set architecture, a vector processor architecture, a very large instruction word architecture. Furthermore, the CPU 2130 can be based on the Von Neuman model or the Harvard model. The CPU 2130 can be a digital signal processor, an FPGA, an ASIC, a PLA, a PLD, or a CPLD. Further, the CPU 2130 can be an x86 processor by Intel or by AMD; an ARM processor, a Power architecture processor by, e.g., IBM; a SPARC architecture processor by Sun Microsystems or by Oracle; or other known CPU architecture.

Referring again to FIG. 22, the data processing system 2200 can include that the SB/ICH 2220 is coupled through a system bus to an I/O Bus, a read only memory (ROM) 2256, universal serial bus (USB) port 2264, a flash binary input/output system (BIOS) 2268, and a graphics controller 2258. PCI/PCIe devices can also be coupled to SB/ICH 2220 through a PCI bus 2262.

The PCI devices may include, for example, Ethernet adapters, add-in cards, and PC cards for notebook computers. The Hard disk drive 2260 and CD-ROM 2256 can use, for example, an integrated drive electronics (IDE) or serial advanced technology attachment (SATA) interface. In one implementation the I/O bus can include a super I/O (SIO) device.

Further, the hard disk drive (HDD) 2260 and optical drive 2266 can also be coupled to the SB/ICH 2220 through a system bus. In one implementation, a keyboard 2270, a mouse 2272, a parallel port 2278, and a serial port 2276 can be connected to the system bus through the I/O bus. Other peripherals and devices that can be connected to the SB/ICH 2220 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes on battery sizing and chemistry or based on the requirements of the intended back-up load to be powered.

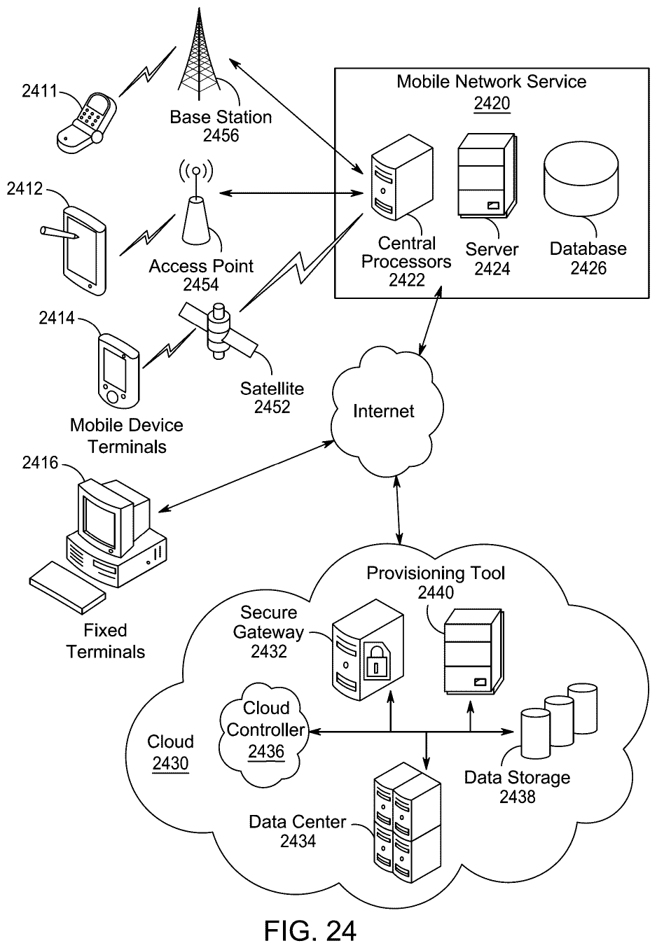

The functions and features described herein may also be executed by various distributed components of a system. For example, one or more processors may execute these system functions, wherein the processors are distributed across multiple components communicating in a network. The distributed components may include one or more client and server machines, which may share processing, as shown by FIG. 24, in addition to various human interface and communication devices (e.g., display monitors, smart phones, tablets, personal digital assistants (PDAs)). More specifically, FIG. 24 illustrates client devices including a smart phone 2411, a tablet 2412, a mobile device terminal 2414 and fixed terminals 2416. These client devices may be commutatively coupled with a mobile network service 2420 via a base station 2456, an access point 2454, a satellite 2452 or via an internet connection. The mobile network service 2420 may comprise central processors 2422, a server 2424 and a database 2426. The fixed terminals 2416 and the mobile network service 2420 may be commutatively coupled via an internet connection to functions in cloud 2430 that may comprise a security gateway 2432, a data center 2434, a cloud controller 2436, a data storage 2438 and a provisioning tool 2440. The network may be a private network, such as the LAN or the WAN, or may be the public network, such as the Internet. Input to the system may be received via direct user input and received remotely either in real-time or as a batch process. Additionally, some implementations may be performed on modules or hardware not identical to those described. Accordingly, other implementations are within the scope that may be disclosed.

The above-described hardware description is a non-limiting example of corresponding structure for performing the functionality described herein.

Numerous modifications and variations of the present disclosure are possible in light of the above teachings. It is therefore to be understood that the invention may be practiced otherwise than as specifically described herein.

The other systems in the comparisons illustrated in FIGS. 2B, 20A, and 20B are listed in Table 8 below.

TABLE 8
Other systems in FIGS. 2B, 20A, and 20B Compared with the CM Based on M-CCIIs

| | |
|---|---|
| Ref-1 | T. Yucehan and E. Yuce, A New Grounded Capacitance Multiplier Using a Single ICFOA and |
| | a Grounded Capacitor, IEEE Transactions on Circuits and Systems-II: Express Briefs, 69 (3) |
| | (2022) 1-5 |
| Ref-2 | V. Stornelli , L. Safari , G. Barile, and G. Ferri, A New Extremely Low Power Temperature |
| | Insensitive Electronically Tunable VCII-Based Grounded Capacitance Multiplier, IEEE |
| | Transactions on Circuits and Systems-II: Express Briefs, 68 (1) (2021) 1-5 |
| Ref-3 | R. Arslanalp and T. Yücehan, “Capacitance multiplier design by using CFOA-,” 23rd Signal |
| | Processing and Communications Applications Conference (SIU), Malatya, Turkey, 2015, pp. |
| | 1393-1396 |
| Ref-4 | P. B. Petrovic, Single VDTA-based Lossless and Lossy Electronically Tunable Positive and |
| | Negative Grounded Capacitance Multipliers, Circuits, Systems, and Signal Processing, 41 |
| | (2022) 1-35 |
| Ref-5 | D. Ozenli, E. Alaybeyoglu, An electronically tunable CMOS implementation of capacitance |
| | multiplier employing CCCDTA, International Journal of Electronics and Communications, 155 |
| | (2022) 1-11 |
| Ref-6 | M. Vahbeh, E. Özer, and F. Kacar, Design of Lossless Negative Capacitance Multiplier |
| | Employing a Single Active Element, electronics, 13 (2024) 1-20 |
| Ref-7 | V. Stornelli, L. Safari, G. Barile , G. Ferri, A new VCII based grounded positive/negative |
| | capacitance multiplier, International Journal of Electronics and Communications, 137 (2021) 1- |
| | 13 |
| Ref-8 | E. Ozer, Electronically tunable CFTA based positive and negative grounded capacitance |
| | multipliers, International Journal of Electronics and Communications, 134 (2021) 1-10. |
| Ref-9 | J. Aguado-Ruiz, A. J. Lopez-Martin, and J. R.Angulo, Three novel improved CMOS C- |
| | multipliers, International Journal of Circuit Theory and Applications, 40 (2010) 1-10. |
| Ref-10 | D. Ozenli, E. Alaybeyoglu, H. Kuntman, A tunable lossy grounded capacitance multiplier |
| | circuit based on VDTA for the low frequency operations, Analog Integrated Circuits and Signal |
| | Processing 113 (2022) 1-8 |
| Ref-11 | New Resistor-Less Electronically Controllable ± C Simulator Employing VCII, DVCC, and a |
| | Grounded Capacitor, Electronics 11 (2022) 1-14 |
| Ref-12 | I. P. Cantoya, and P. M. Furth, Enhanced Grounded Capacitor Multiplier and Its Floating |
| | Implementation for Analog Filters, IEEE Transactions on Circuits and Systems-II: Express |
| | Briefs, 62 (10) (2015) 1-5 |
| Ref-13 | M. A. Al-Absi and A. A. Al-Khulaifi, A New Floating and Tunable Capacitance Multiplier |
| | With Large Multiplication Factor, IEEE Access, 7 (2019) 1-6 |
| Ref-14 | E. Alaybeyog ̌lu, H. Kuntman, Capacitor multiplier with high multiplication factor for |
| | integrated low pass filter of biomedical applications using DTMOS technique, International |
| | Journal of Electronics and Communications, 107 (2019) 1-7 |
| Ref-15 | E. Alaybeyoglu, Implementation of capacitor multiplier with cell-based variable |
| | transconductance amplifier, IET Circuits, Devices & Systems, 13 (3) (2018) 1-6 |
| Ref-16 | B. Sakaci and D. Ozenli, A current mode capacitance multiplier employing a single active |
| | element based on Arbel-Goldminz cells for low frequency applications, Microelectronic |
| | Engineering 288 (2024) 1-14 |
| Ref-17 | B. Sakaci, D. Ozenli and H. H. Kuntman, “An Electronically Tunable Capacitance Multiplier |
| | Employing Single Active Block For The Speech Processing Applications,” 14th International |
| | Conference on Electrical and Electronics Engineering (ELECO), Bursa, Turkiye, 2023, pp. 1-5 |

1. A grounded positive and negative capacitance multiplier, comprising a first number (i) of current conveyors connected in a cascaded manner, wherein
each current conveyor includes a transconductance amplifier (OTA), a feedback network (FBN) including a second number (N) of CMOS inverters, and a feedforward network (FFN) including a third number (M) of CMOS inverters,
a base capacitor is connected between a first input terminal of a first current conveyor of the cascaded current conveyors and a ground node,
a multiplied capacitance is obtained between a second input terminal of the first current conveyor and the ground node, with a capacitance value given by
$${( - \frac{M}{N} )}^{i} * {C}_{B} ,$$
where C_{B} represents a capacitance of the base capacitor,
when i is an odd number, the multiplied capacitance has a negative value, and
when i is an even number, the multiplied capacitance has a positive value.

2. The capacitance multiplier of claim 1, wherein the OTA is a Miller-compensated OTA.

3. The capacitance multiplier of claim 2, wherein the Miller-compensated OTA is further configured to receive a reference current from a bias circuit.

4. The capacitance multiplier of claim 3, wherein the bias circuit includes:
a first stage for generating a complementary to absolute temperature current,
a second stage for generating a curvature-compensation current, and
a third stage for applying a scaling factor to a summation of outputs of the first and second stages.

5. The capacitance multiplier of claim 3, wherein the Miller-compensated OTA includes:
a first stage configured as an NMOS differential input pair with active loads, and
a second stage configured as a common source amplifier, and wherein both the first stage and the second stage are biased by the bias circuit.

6. The capacitance multiplier of claim 5, wherein the reference current is provided from the bias circuit through a CMOS transistor to the first and second stages of the Miller-compensated OTA.

7. The capacitance multiplier of claim 6, wherein the Miller-compensated OTA includes a compensation resistor and a compensation capacitor, and a phase margin of the Miller-compensated OTA is configured to be larger than 65°.

8. The capacitance multiplier of claim 1, wherein each CMOS inverter of the second number (N) of CMOS inverters in the FBN has a pair of NMOS and PMOS transistors as current-limiting devices.

9. The capacitance multiplier of claim 1, wherein each CMOS inverter of the third number (M) of CMOS inverters in the FFN has a pair of NMOS and PMOS transistors as current-limiting devices.

10. The capacitance multiplier of claim 1, wherein i=4, M=15, and N=1.

11. A low-pass filter for processing human biosignals, comprising:
a multiplied capacitance obtained from a base capacitor and a grounded positive and negative capacitance multiplier, the capacitance multiplier including a first number (i) of current conveyors connected in a cascaded manner, wherein
each current conveyor includes a transconductance amplifier (OTA), a feedback network (FBN) including a second number (N) of CMOS inverters, and a feedforward network (FFN) including a third number (M) of CMOS inverters,
the base capacitor is connected between a first input terminal of a first current conveyor of the cascaded current conveyors and a ground node,
the multiplied capacitance is obtained between a second input terminal of the first current conveyor and the ground node, with a capacitance value given by
$${( - \frac{M}{N} )}^{i} * {C}_{B} ,$$
where C_{B} represents a capacitance of the base capacitor,
when i is an odd number, the multiplied capacitance has a negative value, and
when i is an even number, the multiplied capacitance has a positive value.

12. The low-pass filter of claim 11, wherein the OTA is a Miller-compensated OTA.

13. The low-pass filter of claim 12, wherein the Miller-compensated OTA is further configured to receive a reference current from a bias circuit.

14. The low-pass filter of claim 13, wherein the bias circuit includes:
a first stage for generating a complementary to absolute temperature current,
a second stage for generating a curvature-compensation current, and
a third stage for applying a scaling factor to a summation of outputs of the first and second stages.

15. The low-pass filter of claim 13, wherein the Miller-compensated OTA includes:
a first stage configured as an NMOS differential input pair with active loads, and
a second stage configured as a common source amplifier, and wherein both the first stage and the second stage are biased by the bias circuit.

16. The low-pass filter of claim 15, wherein the reference current is provided from the bias circuit through a CMOS transistor to the first and second stages of the Miller-compensated OTA.

17. The low-pass filter of claim 16, wherein the Miller-compensated OTA includes a compensation resistor and a compensation capacitor, and a phase margin of the Miller-compensated OTA is configured to be larger than 65°.

18. The low-pass filter of claim 11, wherein each CMOS inverter of the second number (N) of CMOS inverters in the FBN has a pair of NMOS and PMOS transistors as current-limiting devices.

19. The low-pass filter of claim 11, wherein each CMOS inverter of the third number (M) of CMOS inverters in the FFN has a pair of NMOS and PMOS transistors as current-limiting devices.

20. The low-pass filter of claim 11, wherein the low-pass filter further includes a resistor of 0.5 MΩ, the base capacitor has a capacitance of 1 pF, the capacitance multiplier has a multiplication factor of 50,625, and the low-pass filter has a corner frequency of 6.4 Hz.
